(19) United States
(12) Patent Application Publication
(10) Pub. No.: US 2010/0247688 A1
(43) Pub. Date: Sep. 30, 2010

(54) PIRENZEPINE AND DERIVATIVES THEREOF AS ANTI-AMYLOID AGENTS

(76) Inventors: Andrea Pfeifer, St. Legier (CH); André Schrattenholz, Mainz (DE); Andreas Muhs, Pully (CH)

Correspondence Address: Johnson & Associates, 317A E. Liberty Street, Savannah, GA, 31401, US

(21) Appl. No.: 12/667,445

(22) PCT Filed: Jul. 2, 2008
(86) PCT No.: PCT/EP08/58527

§ 371 Date: May 12, 2010

(30) Foreign Application Priority Data
Jul. 2, 2007 (EP) 07111491.2

(51) Int. Cl. A61K 36/16; C07D 487/14; A61K 31/5513; A61K 38/17; A61K 31/55; A61K 31/714; A61K 31/685; A61P 25/00; A61P 25/28; A61P 31/18; A61P 27/02; A61P 9/00; A61P 5/00; A61P 3/10

(52) U.S. Cl. 424/752; 540/495; 514/220; 514/17.8; 514/213.01; 514/52; 514/78

(57) ABSTRACT

The present invention relates to a compound that is capable of inhibiting the formation of β-amyloid plaques, of reducing and/or retarding the increase the β-amyloid plaque load in the brain of an animal, particularly a mammal, but especially a human. In particular, the invention relates to compounds of formula (I) and to metabolites thereof.

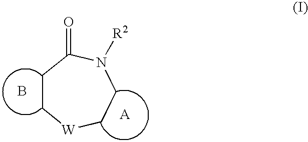

Disclosed herein are compounds particularly compounds related to the pirenzepine family and/or metabolites thereof that are capable of inhibiting the formation of β-amyloid plaques and of reducing the β-amyloid plaque load in the brain of an animal, particularly a mammal, but especially a human. In particular, the invention relates to compounds of the pirenzepine group and to metabolites thereof.

The M1 muscarinic effect of pirenzepine is thought to be responsible for vago-mimetic neuro-humoral regulation potentially useful for treatment of chronic heart failure patients and of patients recovering from myocardial infarction or generally in hypertension.

Pirenzepine has also been implicated in some CNS-related diseases based on its M1 muscarinic inhibitory action, e.g. it is used as a co-medication to antipsychotic drugs. A potential role of muscarinic receptors in schizophrenia is assumed to be the underlying reason.

Pirenzepine is used together with drugs like olanzapine or clozapine to suppress side effects (e.g. emesis or hypersalivation) in cancer or schizophrenia treatments.

Pirenzepine has also been found to be effective in the reduction of progression of myopia, especially in children with promising efficacy results and acceptable safety profile.

Further, pirenzepine has been tested in the treatment of diabetes. Taken together, these studies show that pirenzepine is a relatively safe compound.

A cytoprotective, but particularly a neuroprotective activity of pirenzepine and the pirenzepine metabolite LS-75, is reported in WO 2006/008118.

It was therefore an objective of the present invention to find new therapeutic or diagnostic uses for pirenzepine-type compounds. It was now surprisingly found that these compounds are capable of (a) reducing the β-amyloid plaque load, and/or (b) inhibiting the formation of β-amyloid plaques and/or (c) retarding the increase of amyloid load in tissues and organs, particularly in the brain, of an animal, particularly a mammal, but especially a human, and can thus be used in the treatment of diseases caused by or associated with the formation, accumulation and deposition of amyloid or amyloid-like proteins such as amyloidosis, particularly Alzheimer Disease (AD).

Amyloidosis is not a single disease entity but rather a diverse group of progressive disease processes characterized by extracellular tissue deposits of a waxy, starch-like protein called amyloid, which accumulates in one or more organs or body systems. As the amyloid deposits accumulate, they begin to interfere with the normal function of the organ or body system. There are at least 15 different types of amyloidosis. The major forms are primary amyloidosis without known antecedent, secondary amyloidosis following some other condition, and hereditary amyloidosis.

Secondary amyloidosis occurs during chronic infection or inflammatory disease, such as tuberculosis, a bacterial infection called familial Mediterranean fever, bone infections (osteomyelitis), rheumatoid arthritis, inflammation of the small intestine (granulomatous ileitis), Hodgkin's disease, and leprosy.

Amyloid deposits include amyloid P (pentagonal) component (AP), a glycoprotein related to normal serum amyloid P(SAP), and sulphated glycosaminoglycans (GAG), complex carbohydrates of connective tissue. Amyloid protein fibrils, which account for about 90% of the amyloid material, comprise one of several different types of proteins. These proteins are capable of folding into so-called ‘beta-pleated’ sheet fibrils, a unique protein configuration which exhibits binding sites for Congo red resulting in the unique staining properties of the amyloid protein.

Many diseases of aging are based on or associated with amyloid-like proteins and are characterized, in part, by the buildup of extracellular deposits of amyloid or amyloid-like material that contribute to the pathogenesis, as well as the progression of the disease. These diseases include, but are not limited to, neurological disorders such as Alzheimer's Disease (AD) and diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration, drusen-related optic neuropathy and cataract due to beta-amyloid deposition.

Although pathogenesis of these diseases may be diverse, their characteristic deposits often contain many shared molecular constituents. To a significant degree, this may be attributable to the local activation of pro-inflammatory pathways thereby leading to the concurrent deposition of activated complement components, acute phase reactants, immune modulators, and other inflammatory mediators (McGeer et al., 1994).

Alzheimer's Disease (AD) is a neurological disorder primarily thought to be caused by amyloid plaques, an accumulation of abnormal deposit of proteins in the brain. The most frequent type of amyloid found in the brain of affected individuals is composed primarily of Aβ fibrils. Scientific evidence demonstrates that an increase in the production and accumulation of beta-amyloid protein in plaques leads to nerve cell death, which contributes to the development and progression of AD. Loss of nerve cells in strategic brain areas, in turn, causes reduction in the neurotransmitters and impairment of memory. The proteins principally responsible for the plaque build up include amyloid precursor protein (APP) and two presenilins (presenilin I and presenilin II). Sequential cleavage of the amyloid precursor protein (APP), which is constitutively expressed and catabolized in most cells, by the enzymes β and γ secretase leads to the release of a 39 to 43 amino acid Aβ peptide. The degradation of APPs likely increases their propensity to aggregate in plaques. It is especially the Aβ(1-42) fragment that has a high propensity of building aggregates due to two very hydrophobic amino acid residues at its C-terminus. The Aβ(1-42) fragment is therefore believed to be mainly involved and responsible for the initiation of neuritic plaque formation in AD and to have, therefore, a high pathological potential. Thus a hallmark of AD is the deposition of plaques in the brain of AD patients (Selkoe, 2000; Walsh and Selkoe, 2004). There is therefore a need for agents to prevent the formation of amyloid plaques and to diffuse existing plaques in AD.

Alzheimer's disease (AD) is the most prevalent neurodegenerative disease in the growing population of elderly people. The symptoms of AD manifest slowly and the first symptom may only be mild forgetfulness. In this stage, individuals may forget recent events, activities, the names of familiar people or things and may not be able to solve simple math problems. As the disease progresses, symptoms are more easily noticed and become serious enough to cause people with AD or their family members to seek medical help. Mid-stage symptoms of AD include forgetting how to do simple tasks such as grooming, and problems develop with speaking, understanding, reading, or writing. Later stage AD patients may become anxious or aggressive, may wander away from home and ultimately need total care.

Presently, the only definite way to diagnose AD is to identify plaques and tangles in brain tissue in an autopsy after death of the individual. Therefore, doctors can only make a diagnosis of “possible” or “probable” AD while the person is still alive. Using current methods, physicians can diagnose AD correctly up to 90 percent of the time using several tools to diagnose “probable” AD. Physicians ask questions about the person's general health, past medical problems, and the history of any difficulties the person has carrying out daily activities. Behavioral tests of memory, problem solving, attention, counting, and language provide information on cognitive degeneration and medical tests such as tests of blood, urine, or spinal fluid, and brain scans can provide some further information.

The management of AD consists of medication-based and non-medication based treatments. Treatments aimed at changing the underlying course of the disease (delaying or reversing the progression) have so far been largely unsuccessful. Medicines that restore the deficit (defect), or malfunctioning, in the chemical messengers of the nerve cells (neurotransmitters), in particular the cholinesterase inhibitors (ChEIs) such as tacrine and rivastigmine, have been shown to improve symptoms. ChEIs impede the enzymatic degradation of neurotransmitters thereby increasing the amount of chemical messengers available to transmit the nerve signals in the brain.

For some people in the early and middle stages of the disease, the drugs tacrine (COGNEX®, Morris Plains, N.J.), donepezil (ARICEPT®, Tokyo, JP), rivastigmine (EXELON®, East Hanover, N.J.), or galantamine (REMINYL®, New Brunswick, N.J.) may help prevent some symptoms from becoming worse for a limited time. Another drug, memantine (NAMENDA®, New York, N.Y.), has been approved for treatment of moderate to severe AD. Medications are also available to address the psychiatric manifestations of AD. Also, some medicines may help control behavioral symptoms of AD such as sleeplessness, agitation, wandering, anxiety, and depression. Treating these symptoms often makes patients more comfortable and makes their care easier for caregivers. Unfortunately, despite significant treatment advances showing that this class of agents is consistently better than a placebo, the disease continues to progress, and the average effect on mental functioning has only been modest. Many of the drugs used in AD medication such as, for example, ChEIs also have side effects that include gastrointestinal dysfunction, liver toxicity and weight loss.

Another disease that is based on or associated with the accumulation and deposit of amyloid-like protein is macular degeneration.

Macular degeneration is a common eye disease that causes deterioration of the macula, which is the central area of the retina (the paper-thin tissue at the back of the eye where light-sensitive cells send visual signals to the brain). Sharp, clear, ‘straight ahead’ vision is processed by the macula. Damage to the macula results in the development of blind spots and blurred or distorted vision. Age-related macular degeneration (AMD) is a major cause of visual impairment in the United States and for people over age 65 it is the leading cause of legal blindness among Caucasians. Approximately 1.8 million Americans age 40 and older have advanced AMD, and another 7.3 million people with intermediate AMD are at substantial risk for vision loss. The government estimates that by 2020 there will be 2.9 million people with advanced AMD. Victims of AMD are often surprised and frustrated to find out how little is known about the causes and treatment of this blinding condition.

There are two forms of macular degeneration: dry macular degeneration and we macular degeneration. The dry form, in which the cells of the macula slowly begin to break down, is diagnosed in 85 percent of macular degeneration cases. Both eyes are usually affected by dry AMD, although one eye can lose vision while the other eye remains unaffected. Drusen, which are yellow deposits under the retina, are common early signs of dry AMD. The risk of developing advanced dry AMD or wet AMD increases as the number or size of the drusen increases. It is possible for dry AMD to advance and cause loss of vision without turning into the wet form of the disease; however, it is also possible for early-stage dry AMD to suddenly change into the wet form.

The wet form, although it only accounts for 15 percent of the cases, results in 90 percent of the blindness, and is considered advanced AMD (there is no early or intermediate stage of wet AMD). Wet AMD is always preceded by the dry form of the disease. As the dry form worsens, some people begin to have abnormal blood vessels growing behind the macula. These vessels are very fragile and will leak fluid and blood (hence ‘wet’ macular degeneration), causing rapid damage to the macula.

The dry form of AMD will initially often cause slightly blurred vision. The center of vision in particular may then become blurred and this region grows larger as the disease progresses. No symptoms may be noticed if only one eye is affected. In wet AMD, straight lines may appear wavy and central vision loss can occur rapidly.

Diagnosis of macular degeneration typically involves a dilated eye exam, visual acuity test, and a viewing of the back of the eye using a procedure called fundoscopy to help diagnose AMD, and—if wet AMD is suspected—fluorescein angiography may also be performed. If dry AMD reaches the advanced stages, there is no current treatment to prevent vision loss. However, a specific high dose formula of antioxidants and zinc may delay or prevent intermediate AMD from progressing to the advanced stage. Macugen® (pegaptanib sodium injection), laser photocoagulation and photodynamic therapy can control the abnormal blood vessel growth and bleeding in the macula, which is helpful for some people who have wet AMD; however, vision that is already lost will not be restored by these techniques. If vision is already lost, low vision aids exist that can help improve the quality of life.

One of the earliest signs of age-related macular degeneration (AMD) is the accumulation of extracellular deposits known as drusen between the basal lamina of the retinal pigmented epithelium (RPE) and Bruch's membrane (BM). Recent studies conducted by Anderson et al. have confirmed that drusen contains amyloid beta. (Experimental Eye Research 78 (2004) 243-256).

Ongoing research continues with studies exploring environmental, genetic, and dietary factors that may contribute to AMD. New treatment strategies are also being explored, including retinal cell transplants, drugs that will prevent or slow down the progress of the disease, radiation therapy, gene therapies, a computer chip implanted in the retina that may help stimulate vision and agents that will prevent the growth of new blood vessels under the macula.

An important factor to consider when developing new drugs is the ease of use for the target patients. Oral drug delivery—specifically tablets, capsules and softgels—account for 70% of all dosage forms consumed because of patient convenience. Drug developers agree that patients prefer oral delivery rather than subjecting themselves to injections or other, more invasive forms of medicinal administration. Formulations resulting in low dosing intervals (i.e. once a day or sustained release) are also preferable. The ease of administering antibiotics in oral dosage forms results in an increase of patient compliance during treatment.

What is needed are effective methods and compositions for preventing or addressing the complications associated with amyloidosis, a group of diseases and disorders associated with amyloid plaque formation such as Alzheimer's Disease. In particular what is needed are agents capable of counteracting the physiological manifestations of the disease such as the formation of plaques associated with aggregation of fibers of the amyloid or amyloid-like peptide.

Thus, a first aspect of the present invention relates to a compound of formula I

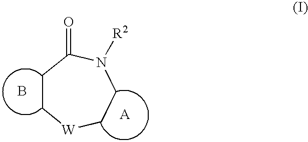

wherein A and B are five- or six-membered rings optionally containing at least one heteroatom selected from N, S and O, wherein the rings are optionally mono- or polysubstituted with halo, e.g. F, Cl, Br, or I, C₁-C₄-(halo)-alkyl, C₁-C₄-(halo)-alkoxy, amino, C₁-C₄-alkyl-amino, or di(C₁-C₄-alkyl)amino,

### W is S, O, NR¹ or CHR¹

R¹ is hydrogen, Y or COY,
R² is hydrogen or C₁-C₄-(halo)-alkyl, and
Y is C₁-C₆ (halo)alkyl, or C₃-C₈ cyclo-(halo)alkyl, wherein the alkyl or cycloalkyl group is optionally substituted with a five- or six-membered ring optionally containing at least one heteroatom selected from N, S and O, and wherein the ring is optionally mono- or poly-substituted with halo, C₁-C₄-(halo)alkyl, C₁-C₄(halo)alkoxy, amino, C₁-C₄-alkyl amino, di(C₁-C₄-alkyl)amino or Z,
wherein Z is a C₁-C₆ (halo) alkyl group ω-substituted with a group N(R⁴)₂, wherein each R⁴ is independently hydrogen, C₁-C₈ alkyl, or CO—C₁-C₈-alkyl or wherein both R⁴ together from a five- or six-membered ring optionally containing at least one further heteroatom selected from N, S and O, wherein the ring is optionally mono- or polysubstituted with halo, C₁-C₄(halo)-alkyl and C₁-C₄(halo)alkoxy, or of a salt or derivative thereof, including pharmaceutically effective metabolites thereof, or to the use thereof, for
(a) reducing the β-amyloid plaque load, and/or (b) inhibiting the formation of β-amyloid plaques and/or (c) retarding the increase of amyloid load in tissues and organs of an animal, particularly a mammal, but especially a human, but particularly in the brain of an animal, particularly a mammal, but especially a human.

The term “(halo)alkyl” as used above in the characterization of a compound of formula I is meant within the scope of the present invention to refer to an alkyl group which optionally contains at least one halo, e.g. F, Cl, Br or I substituent up to perhalogenation.

The term “salt” is meant to refer to pharmaceutically acceptable salts of compounds of formula I with suitable cations and/or anions. Examples of suitable cations are alkaline metal cations such as Li⁺; Na⁺ and K⁺, alkaline earth metal cations such as Mg⁺ and Ca⁺ as well as suitable organic cations, e.g. ammoniums or substituted ammonium cations. Examples of pharmaceutically acceptable anions are inorganic anions such as chloride, sulfate, hydrogen sulfate, phosphate or organic cations such as acetate, citrate, tartrate, etc.

Derivatives of compounds of formula I are any molecules which are converted under physiological conditions to a compound of formula I, e.g. esters, amides etc. of compounds of formula I or molecules which are products of metabolization reactions of a compound of formula I such as, for example, the compound of formula III.

In the compounds of formula I, the cyclic groups A and B are particularly selected from

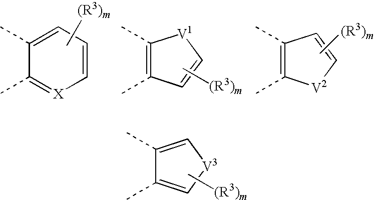

wherein X is N or CR³,
V¹, V² or V³ are selected from —O—, —S—, and NR⁶,
R³ is in each case independently halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino,
m is an integer of 0-2, and
R⁶ is hydrogen or C₁-C₄-(halo)alkyl.

More particularly, the cyclic group A is selected from

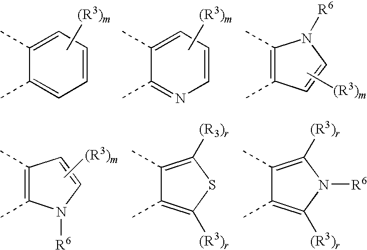

wherein R³ is defined as above,
m is an integer of 0-2,
r is an integer of 0-1 and
R⁶ is hydrogen or methyl.

More preferably, the cyclic group B is selected from

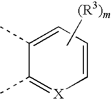

wherein X, R³ and m are as defined above

in one embodiment R¹ is Y. In this case Y is preferably C₃-C₈ cyclo(halo)-alkyl, e.g. cyclopropyl, cyclobutyl or cyclopentyl.

In a further embodiment, R¹ is COY and Y is —(CHR7)q-R⁸

wherein R⁷ is hydrogen, halo or C₁-C₄-(halo)alkyl,
q is an integer of 1-4, and preferably 1 and
R⁸ is a five- or six-membered ring optionally containing at least one heteroatom, wherein the ring is optionally mono- or polysubstituted with C₁-C₄(halo)alkyl or a ω-amino-substituted alkyl group Z as defined above.

In this embodiment, R⁸ is particularly selected from

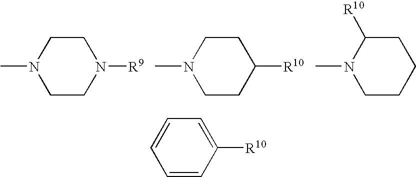

wherein R⁹ is hydrogen or C₁-C₄(halo)alkyl and R¹⁰ is a ω-amino-substituted alkyl group Z as defined above.

R⁹ is particularly a methyl group. The ω-amino-substituted alkyl group Z is preferably a C₁-C₄(halo)alkyl group having a terminal amino group which is substituted with at least one C₁-C₆ alkyl group, e.g. a diethylamino, or di-isobutylamino group, or with a CO(C₁-C₆ alkyl group and with hydrogen or a C₁-C₂ alkyl group.

In a specific embodiment, the cyclic group A and B is

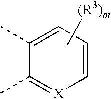

wherein X is N or CR³,
R³ is in each case independently halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2

In another specific embodiment, the cyclic group A is

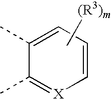

wherein X is N
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2.

In another specific embodiment, the cyclic group B is

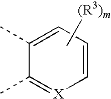

wherein X is CH
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2.

In another specific embodiment, the cyclic group A is

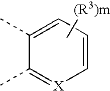

wherein X is N
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2; and
wherein the cyclic group B is

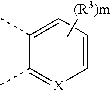

wherein X is CH
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2.

In still another specific embodiment, the invention relates to a compound of formula I as defined herein above, wherein

### W is NR¹

### R¹ is COY and

Y is —(CHR⁷)q-R⁸
wherein R⁷ is hydrogen, halo or C₁-C₄-(halo)alkyl,
q is an integer of 1-4, and preferably 1 and
R⁸ is a five- or six-membered ring optionally containing at least one heteroatom, wherein the ring is optionally mono- or polysubstituted with C₁-C₄(halo)alkyl or a ω-amino-substituted alkyl group Z as defined above.

In another specific embodiment, the cyclic group A is

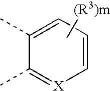

wherein X is N
R³ is halo, C₁-C₄-(halo)-alkyl, C₁-C₄-(halo)-alkoxy, amino, C₁-C₄-alkyl-amino, or di(C₁-C₄-alkyl)amino, and
m is an integer of 0-2; and wherein the cyclic group B is

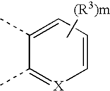

wherein X is CH
R³ is halo, C₁-C₄-(halo)-alkyl, C₁-C₄-(halo)-alkyl, C₁-C₄-(halo)-alkoxy, amino, C₁-C₄ alkyl-amino, or di(C₁-C₄-alkyl)amino, and
m is an integer of 0-2; and wherein

### W is NR¹

### R¹ is COY and

Y is —(CHR⁷)q-R⁸
wherein R⁷ is hydrogen, halo or C₁-C₄-(halo)alkyl,
q is an integer of 1-4, and preferably 1 and
R⁸ is a five- or six-membered ring optionally containing at least one heteroatom, wherein the ring is optionally mono- or polysubstituted with C₁-C₄(halo)alkyl or a ω-amino-substituted alkyl group Z as defined above.

In another specific embodiment, the cyclic group A is

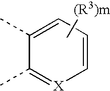

wherein X is N
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2; and
wherein the cyclic group B is

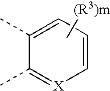

wherein X is CH
R³ is halo, C₁-C₄-(halo)-alkyl, C₁-C₄-(halo)-alkyl, C₁-C₄-(halo)alkoxy, amino, C₁-C₄-alkyl-amino, or di(C₁-C₄-alkyl)amino, and
m is an integer of 0-2; and wherein

### W is NR¹

### R¹ is COY and

Y is —(CHR⁷)q-R⁸
wherein R⁷ is hydrogen or C₁-C₄-alkyl,
q is an integer of 1-4, and preferably 1 and
R⁸ is a six-membered ring containing at least one N, wherein the ring is mono- or polysubstituted with C₁-C₄(halo)alkyl.

In a specific embodiment, the invention relates to a compound of formula I as defined herein above, wherein

### W is NR¹

R¹ is hydrogen
the cyclic group A and B is

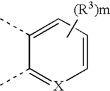

wherein X is N or CR³,
R³ is in each case independently halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2

In another specific embodiment, the invention relates of a compound of formula I as defined herein above, wherein

### W is NR¹

R¹ is hydrogen
the cyclic group A is

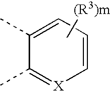

wherein X is N
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2.

In another specific embodiment, the invention relates to a compound of formula I as defined herein above, wherein

### W is NR¹

R¹ is hydrogen
the cyclic group B is

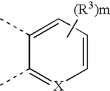

wherein X is CH
R³ is halo, C₁-C₄-(halo)alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2.

In another specific embodiment, the invention relates to a compound of formula I as defined herein above, wherein

### W is NR¹

R¹ is hydrogen
the cyclic group A is

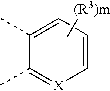

wherein X is N
R³ is halo, C₁-C₄-(halo)-alkyl, C₁-C₄-(halo)-alkyl, C₁-C₄-(halo)-alkoxy, amino, C₁-C₄-alkyl-amino, or di(C₁-C₄-alkyl)amino, and
m is an integer of 0-2; and
wherein the cyclic group B is

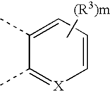

wherein X is CH
R³ is halo, C₁-C₄-(halo)-alkyl, C₁-C₄-(halo)-alkyl C₁-C₄-(halo)-alkoxy, amino, C₁-C₄-alkylamino, or di(C₁-C₄-alkyl)amino, and
m is an integer of 0-2.

In another specific embodiment, the invention relates to a compound of formula I as defined herein above, wherein

### W is NR¹

R¹ is hydrogen
the cyclic group A is

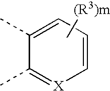

wherein X is N
R³ is C₁-C₄-(halo)-alkyl, and
m is an integer of 0-2; and
wherein the cyclic group B is

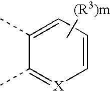

wherein X is CH
R³ is in each case C₁-C₄-(halo)-alkyl, and
m is an integer of 0-2.

Specific examples of compounds of formula I are pirenzepine and related compounds as disclosed in FR 1,505,795, U.S. Pat. Nos. 3,406,168, 3,660,380, 4,021,557, 4,210,648, 4,213,984, 4,213,985, 4,277,399, 4,308,206, 4,317,823, 4,335,250, 4,424,222, 4,424,226, 4,724,236, 4,863,920, 5,324,832, 5,620,978, 6,316,423, otenzepad and related compounds as disclosed in U.S. Pat. Nos. 3,406,168, 5,324,832 and 5,712,269, AQ-RA741 and related compounds as disclosed in U.S. Pat. Nos. 5,716,952, 5,576,436 and 5,324,832, viramune and related compounds as disclosed in EP-A-0429987 and U.S. Pat. Nos. 5,366,972, 5,705,499, BIBN 99 and related compounds as disclosed in U.S. Pat. Nos. 6,022,683 and 5,935,781, DIBD, telenzepine and related compounds as disclosed in EP-A-0035519, and U.S. Pat. No. 4,381,301 and salts or derivatives thereof. The above documents are herein incorporated by reference.

Further preferred compounds are 7-azabicyclo-[2.2.1]-heptane and heptene compounds such as a tiotropium bromide as disclosed in U.S. Pat. Nos. 5,817,679, 6,060,473, 6,077,846, 6,117,889, 6,255,490, 6,403,584, 6,410,583, 6,537,524, 6,579,889, 6,608,055, 6,627,644, 6,635,658, 6,693,202, 6,699,866 and 6,756,392, heterocyclic compounds, e.g. pyrrolidinones, tetrahydropyridines, isoxazocarboxamides, thienopyrane carboxamides, or benzopyranes, such as alvameline tartrate and related compounds, disclosed in U.S. Pat. Nos. 6,306,861, 6,365,592, 6,403,594, 6,486,163, 6,528,529, 6,680,319, 6,716,857 and 6,759,419, metoclopramide and related compounds as disclosed in U.S. Pat. No. 3,177,252 and QNB and related compounds as disclosed in U.S. Pat. No. 2,648,667 and salts and derivatives thereof. The above documents are herein incorporated by reference.

In a specific embodiment, the present invention relates to a compound of formula including pharmaceutically effective metabolites thereof, according to the invention and as defined herein, or a pharmaceutical composition comprising said compound and/or said pharmaceutically effective metabolites thereof, or to the use thereof, for

(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs of an animal, particularly a mammal, but especially a human, but particularly in the brain of an animal, particularly a mammal, but especially a human.

By reducing the β-amyloid plaque load, inhibiting the formation of β-amyloid plaques and/or retarding the increase of amyloid load in the brain of an animal, particularly a mammal, but especially a human, the effect of a disease or condition caused by or associated with the formation and deposition of β-amyloid plaques in tissues and organs, but particularly in the brain, of an animal, particularly a mammal, but especially a human, can be reduced and/or ameliorated.

Accordingly, in a specific embodiment, the present invention relates to a compound of formula I, including pharmaceutically effective metabolites thereof, according to the invention and as defined herein, or a pharmaceutical composition comprising said compound and/or said pharmaceutically effective metabolites thereof, or to the use thereof, for the treatment of a disease or disorder caused by or associated with the formation, accumulation and deposition of amyloid or amyloid-like proteins by

(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs of an animal, particularly a mammal, but especially a human, but particularly in the brain of an animal, particularly a mammal, but especially a human.

Accordingly, in one embodiment, the invention relates to a compound of formula I, including pharmaceutically effective metabolites thereof, according to the invention and as further defined herein or a pharmaceutical composition comprising said compound and/or a pharmaceutically effective metabolites thereof, or the use thereof, for the treatment of a disease or condition in an animal, particularly a mammal, but especially a human, which is caused by or associated with the formation of β-amyloid plaques in tissues and organs, but particularly in the brain, of an animal, particularly a mammal, but especially a human, particularly a diseases or condition selected from the group consisting of neurological disorders such as Alzheimer's Disease (AD) and diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration, drusen-related optic neuropathy and cataract due to beta-amyloid deposition, but especially Alzheimer's disease, by

(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more;
in tissues and organs, but particularly in the brain, of an animal, particularly a mammal, but especially a human.

In one embodiment, the invention relates to a compound of formula I, including pharmaceutically effective metabolites thereof, according to the invention and as further defined herein or a pharmaceutical composition comprising said compound and/or said pharmaceutically effective metabolites thereof, or to the use thereof, for retaining or increasing cognitive memory capacity but, particularly, for restoring the cognitive memory capacity of an animal, particularly a mammal or a human, suffering from memory impairment.

It is a further object of the invention to provide a therapeutic composition, and a method of producing such a composition, comprising a compound of formula I according to the invention and as further defined herein and/or a pharmaceutically effective metabolite thereof for retaining or increasing cognitive memory capacity but, particularly, for restoring the cognitive memory capacity of an animal, particularly a mammal or a human, suffering from memory impairment.

In one embodiment, the invention provides a method of (a) reducing the β-amyloid plaque load, (b) inhibiting the formation of β-amyloid plaques and/or (c) retarding the increase of amyloid load in tissues and organs, but particularly in the brain, of an animal, particularly a mammal, but especially a human, by administering to an animal, particularly a mammal, but especially a human, a compound of formula I and/or pharmaceutically effective metabolites thereof according to the invention and as further defined herein or a pharmaceutical composition comprising said compound and/or a pharmaceutically effective metabolites thereof.

In one embodiment, the invention relates to a method of

(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human, by administering to an animal, particularly a mammal, but especially a human a compound of formula I according to the invention and as further defined herein and/or a pharmaceutically effective metabolite thereof or a pharmaceutical composition comprising said compound and/or a pharmaceutically effective metabolite thereof.

In one embodiment, the invention provides a method for treating in an animal, particularly a mammal, but especially a human, a condition caused by or associated with the formation of β-amyloid plaques in tissues and organs, but particularly in the brain, of an animal, particularly a mammal, but especially a human and resulting in an increased plaque load by

(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or,
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human, through administration of a compound of formula I according to the invention and as further defined herein and/or a pharmaceutically effective metabolites thereof or a pharmaceutical composition comprising said compound and/or a pharmaceutically effective metabolites thereof.

In particular, said condition caused by or associated with the formation of β-amyloid plaques in tissues and organs, but particularly in the brain, of an animal, particularly a mammal, but especially a human and resulting in an increased plaque load is selected from the group consisting of neurological disorders such as Alzheimer's Disease (AD) and diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration, drusen-related optic neuropathy and cataract due to beta-amyloid deposition, but especially Alzheimer's disease.

In a specific embodiment the invention provides a method for retaining or increasing cognitive memory capacity but, particularly, for restoring the cognitive memory capacity of an animal, particularly a mammal or a human, suffering from memory impairment by administering to an animal, particularly a mammal or a human, a compound of formula I according to the invention and as further defined herein and/or a pharmaceutically effective metabolite thereof or a pharmaceutical composition comprising said compound and/or a pharmaceutically effective metabolites thereof.

In another embodiment, the invention relates to the treatment of an animal, particularly a mammal or a human, suffering from an amyloid-associated condition characterized by a loss of cognitive memory capacity with a therapeutic composition comprising a compound of formula I according to the invention and as further defined herein and/or a pharmaceutically effective metabolite thereof, which treatment leads to the retention of cognitive memory capacity and/or an increase in cognitive memory capacity and/or a restoration of cognitive memory capacity in an animal, particularly a mammal or a human.

In one aspect of the invention, a compound of formula I

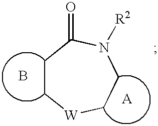

wherein A and B are five- or six-membered rings optionally containing at least one heteroatom selected from N, S and O, wherein the rings are optionally mono- or polysubstituted with halo, e.g. F, Cl, Br, or I, C₁-C₄(halo)-alkyl, C₁-C₄-(halo)-alkoxy, amino, C₁-C₄-alkyl-amino, or di(C₁-C₄-alkyl)amino,

### W is S, O, NR¹ or CHR¹

R¹ is hydrogen, Y or COY,
R² is hydrogen or C₁-C₄-(halo)-alkyl, and
Y is C₁-C₆ (halo)alkyl, or C₃-C₈ cyclo-(halo)alkyl, wherein the alkyl or cycloalkyl group is optionally substituted with a five- or six-membered ring optionally containing at least one heteroatom selected from N, S and O, and wherein the ring is optionally mono- or poly-substituted with halo, C₁-C₄-(halo)alkyl, C₁-C₄-(halo)alkoxy, amino, C₁-C₄-alkyl amino, di(C₁-C₄-alkyl)amino or Z,
wherein Z is a C₁-C₆-(halo) alkyl group ω-substituted with a group N(R4)₂, wherein each R⁴ is independently hydrogen, C₁-C₈ alkyl, or CO—C₁-C₈-alkyl or wherein both R⁴ together from a five- or six-membered ring optionally containing at least one further heteroatom selected from N, S and O, wherein the ring is optionally mono- or polysubstituted with halo, C₁-C₄-(halo)-alkyl and C₁-C₄-(halo)alkoxy, or of a salt or derivative thereof, including pharmaceutically effective metabolites thereof,
is used for (a) reducing the β-amyloid plaque load, (b) inhibiting the formation of β-amyloid plaques and/or (c) retarding the increase of amyloid load in tissues and organs of an animal, particularly a mammal, but especially a human, but particularly in the brain of an animal, particularly a mammal, but especially a human.

In particular, the compound of formula I and/or a pharmaceutically effective metabolite thereof according to the invention is used for the treatment of a condition caused by or associated with the formation of β-amyloid plaques in tissues and organs, but particularly in the brain, of an animal, particularly a mammal, but especially a human and resulting in an increased plaque load selected from the group consisting of neurological disorders such as Alzheimer's Disease (AD) and diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeldt Jacob disease, Parkinson's disease, HIV-related dementia, ALS (amyotropic lateral sclerosis), Adult Onset Diabetes; senile cardiac amyloidosis; endocrine tumors, and others, including macular degeneration, drusen-related optic neuropathy and cataract due to beta-amyloid deposition, drusen-related optic neuropathy and cataract due to beta-amyloid deposition, but especially Alzheimer's disease.

In one embodiment, the compound of formula I and/or a pharmaceutically effective metabolite thereof according to the invention is used for the treatment of an animal, particularly a mammal or a human, suffering from an amyloid-associated condition characterized by a loss of cognitive memory capacity with a therapeutic composition comprising a compound of formula I according to the invention and as further defined herein and/or a pharmaceutically effective metabolite thereof, which treatment leads to the retention of cognitive memory capacity and/or an increase in cognitive memory capacity and/or a restoration of cognitive memory capacity in an animal, particularly a mammal or a human.

Further, the invention encompasses compounds which are metabolized to give diaryl diazepinones according to formula I such as clozepine and olenzepine.

In one embodiment, the invention relates to a compound of formula II

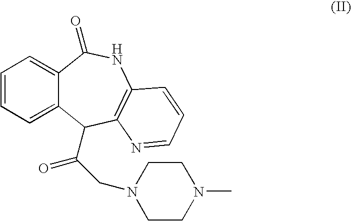

and/or a pharmaceutically effective metabolite thereof or a pharmaceutical composition comprising said compound and/or a pharmaceutically effective metabolites thereof, or to the use thereof, for
(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, particularly in the brain, of an animal, particularly a mammal, but especially a human, particularly in form of a pharmaceutical composition together with one or more pharmaceutically acceptable diluents or carriers therefore.

In one embodiment, the invention relates to a compound of formula II

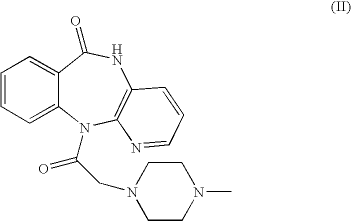

and/or a pharmaceutically effective metabolite thereof or a pharmaceutical composition comprising said compound and/or a pharmaceutically effective metabolites thereof, or to the use thereof, for the treatment of a disease or disorder caused by or associated with the formation, accumulation and deposition of amyloid or amyloid-like proteins by administering to an animal, particularly a mammal or a human, a compound of formula II and/or a pharmaceutically effective metabolite thereof or a pharmaceutical composition comprising said compound and/or a pharmaceutically effective metabolites thereof.

In one embodiment, the invention relates to a compound of formula II

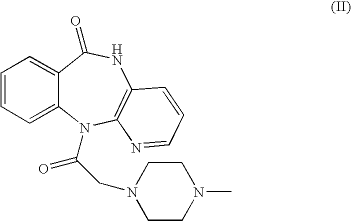

and/or a pharmaceutically effective metabolite thereof or a pharmaceutical composition comprising said compound and/or a pharmaceutically effective metabolites thereof, or to the use thereof, for retaining or increasing cognitive memory capacity but, particularly for restoring the cognitive memory capacity of an animal, particularly a mammal or a human, suffering from memory impairment by administering to an animal, particularly a mammal or a human, a compound of formula II and/or a pharmaceutically effective metabolite thereof or a pharmaceutical composition comprising said compound and/or a pharmaceutically effective metabolites thereof.

It is a further object of the invention to provide a therapeutic composition, and a method of producing such a composition, comprising a compound of formula II according to the invention and as further defined herein and/or a pharmaceutically effective metabolite thereof for retaining or increasing cognitive memory capacity but, particularly, for restoring the cognitive memory capacity of an animal, particularly a mammal or a human, suffering from memory impairment.

In one specific embodiment, the invention relates to a compound of formula II

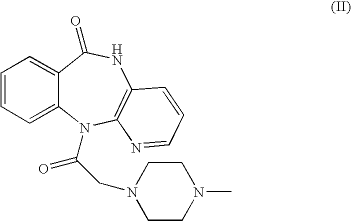

and/or a pharmaceutically effective metabolite thereof or a pharmaceutical composition comprising said compound and/or a pharmaceutically effective metabolite thereof, for the treatment in an animal, particularly a mammal, but especially a human of a condition caused by or associated with the formation of β-amyloid plaques in tissues and organs, but particularly in the brain, and resulting in an increased plaque load, or for the manufacture of a medicament for use in such a treatment, by
(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or,
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human, particularly in form of a pharmaceutical composition together with one or more pharmaceutically acceptable diluents or carriers therefore.

In one embodiment, the invention relates to a compound of formula II, including pharmaceutically effective metabolites thereof, according to the invention and as further defined herein or a pharmaceutical composition comprising said compound and/or a pharmaceutically effective metabolites thereof, or to the use thereof, for the treatment of a disease or condition in an animal, particularly a mammal, but especially a human, which is caused by or associated with the formation of β-amyloid plaques in tissues and organs, but particularly in the brain, of an animal, particularly a mammal, but especially a human, particularly a diseases or condition selected from the group consisting of neurological disorders such as Alzheimer's Disease (AD) and diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration, drusen-related optic neuropathy and cataract due to beta-amyloid deposition, but especially Alzheimer's disease, by

(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 56% and up to 60% or more;
in tissues and organs, but particularly in the brain, of an animal, particularly a mammal, but especially a human.

In one specific embodiment, the invention relates to a compound of formula II

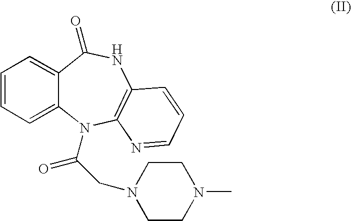

and/or a pharmaceutically effective metabolite thereof or a pharmaceutical composition comprising said compound and/or a pharmaceutically effective metabolite thereof, or to the use thereof, for the treatment in an animal, particularly a mammal, but especially a human suffering from an amyloid-associated condition characterized by a loss of cognitive memory capacity with a compound of formula II and/or a pharmaceutically effective metabolite thereof or a pharmaceutical composition comprising said compound and/or a pharmaceutically effective metabolite thereof, which treatment leads to the retention of cognitive memory capacity and/or an increase in cognitive memory capacity and/or a restoration of cognitive memory capacity in an animal, particularly a mammal or a human.

In particular, the invention relates to the treatment of an animal, particularly a mammal or a human, suffering from an amyloid-associated condition characterized by a loss of cognitive memory capacity with a therapeutic composition comprising a compound of formula II according to the invention and as further defined herein and/or a pharmaceutically effective metabolite thereof, which treatment leads to the retention of cognitive memory capacity and/or an increase in cognitive memory capacity and/or a restoration of cognitive memory capacity in an animal, particularly a mammal or a human.

In one embodiment, the invention provides a method of (a) reducing the β-amyloid plaque load, (b) inhibiting the formation of β-amyloid plaques and/or (c) retarding the increase of amyloid load in tissues and organs, but particularly in the brain, of an animal, particularly a mammal, but especially a human by administering to an animal, particularly a mammal, but especially a human a compound of formula II according to the invention and as described herein before and/or a pharmaceutically effective metabolite thereof or a pharmaceutical composition comprising said compound and/or a pharmaceutically effective metabolites thereof.

In one embodiment, the invention relates to a method of

(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human by administering to an animal, particularly a mammal, but especially a human a compound of formula II according to the invention and as described herein before, and/or a pharmaceutically effective metabolite thereof or a pharmaceutical composition comprising said compound and/or a pharmaceutically effective metabolites thereof.

In one embodiment, the invention provides a method for treating in an animal, particularly a mammal, but especially, a human a condition caused by or associated with the formation of β-amyloid plaques in tissues and organs, but particularly in the brain, and resulting in an increased plaque load by

(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or,
(b) inhibiting the formation of β-amyloid' plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human through administration of a compound of formula II according to the invention and as described herein before and/or a pharmaceutically effective metabolite thereof or a pharmaceutical composition comprising said compound and/or a pharmaceutically effective metabolites thereof.

In one embodiment, the invention provides a method for retaining or increasing cognitive memory capacity but, particularly, for restoring the cognitive memory capacity of an animal, particularly a mammal or a human, suffering from memory impairment by administering to an animal, particularly a mammal or a human, a compound of formula II according to the invention and as further defined herein and/or a pharmaceutically effective metabolite thereof or a pharmaceutical composition comprising said compound and/or a pharmaceutically effective metabolites thereof.

In a specific embodiment of the invention, the compound of formula I as disclosed herein before, but particularly a compound of formula II, or a pharmaceutical composition comprising said compound in a pharmaceutically effective amount, is administered orally.

In another specific embodiment of the invention, the compound of formula I but particularly formula II or a pharmaceutical composition comprising said compound in a pharmaceutically effective amount, is used as a pro-drug.

In one embodiment, the invention relates to a compound of formula III

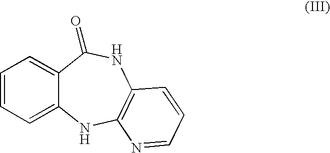

or a pharmaceutical composition comprising said compound in a pharmaceutically effective amount, or to the use thereof; for
(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human, particularly in form of a pharmaceutical composition together with one or more pharmaceutically acceptable diluents or carriers therefore

In one embodiment, the invention relates to a compound of formula III

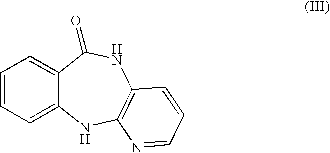

or a pharmaceutical composition comprising said compound in a pharmaceutically effective amount, or to the use thereof, for the treatment of a disease or disorder caused by or associated with the formation, accumulation and deposition of amyloid or amyloid-like proteins by administering to an animal, particularly a mammal or a human, a compound of formula III or a pharmaceutical composition comprising said compound.

In one embodiment, the invention relates to a compound of formula III

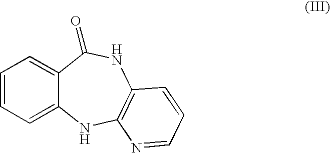

or a pharmaceutical composition comprising said compound in a pharmaceutically effective amount, or to the use thereof, for retaining or increasing cognitive memory capacity but, particularly for restoring the cognitive memory capacity of an animal, particularly a mammal or a human, suffering from memory impairment by administering to an animal, particularly a mammal or a human, a compound of formula III or a pharmaceutical composition comprising said compound in a pharmaceutically effective amount.

In one embodiment, the invention relates to a compound of formula in

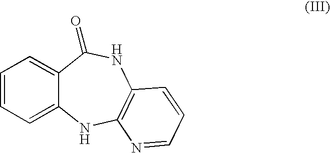

or a pharmaceutical composition comprising said compound in a pharmaceutically effective amount, or to the use thereof, for the treatment in an animal, particularly a mammal, but especially a human of a condition caused by or associated with the formation of β-amyloid plaques in tissues and organs, but particularly in the brain, and resulting in an increased plaque load, or for the manufacture of a medicament for use in such a treatment, by
(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or,
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in the brain of an animal, particularly a mammal, but especially a human, particularly in form of a pharmaceutical composition together with one or more pharmaceutically acceptable diluents or carriers therefore.

In one embodiment, the invention relates to a compound of formula III

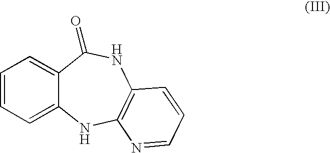

or a pharmaceutical composition comprising said compound in a pharmaceutically effective amount, or to the use thereof, for the treatment in an animal, particularly a mammal, but especially a human suffering from an amyloid-associated condition characterized by a loss of cognitive memory capacity with a compound of formula III or a pharmaceutical composition comprising said compound in a pharmaceutically effective amount, which treatment leads to the retention of cognitive memory capacity and/or an increase in cognitive memory capacity and/or a restoration of cognitive memory capacity in an animal, particularly a mammal or a human.

It is a further object of the invention to provide a therapeutic composition, and a method of producing such a composition, comprising a compound of formula III according to the invention and as further defined herein for retaining or increasing cognitive memory capacity but, particularly, for restoring the cognitive memory capacity of an animal, particularly a mammal or a human, suffering from memory impairment.

In one embodiment, the invention provides a method of (a) reducing the β-amyloid plaque load, (b) inhibiting the formation of β-amyloid plaques and/or (c) retarding the increase of amyloid load in tissues and organs, but particularly in the brain, of an animal, particularly a mammal, but especially a human by administering to an animal, particularly a mammal, but especially a human a compound of formula III according to the invention and as described herein before or a pharmaceutical composition comprising said compound in a pharmaceutically effective amount.

In one embodiment, the invention relates to a method of

(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human by administering to an animal, particularly a mammal, but especially a human a compound of formula III according to the invention and as described herein before or a pharmaceutical composition comprising said compound in a pharmaceutically effective amount.

In another specific embodiment, the invention provides a method for retaining or increasing cognitive memory capacity but, particularly, for restoring the cognitive memory capacity of an animal, particularly a mammal or a human, suffering from memory impairment by administering to an animal, particularly a mammal or a human, a compound of formula III according to the invention and as further defined herein or a pharmaceutical composition comprising said compound.

In one embodiment, the invention provides a method for treating in an animal, particularly a mammal, but especially a human a condition caused by or associated with the formation of β-amyloid plaques in tissues and organs, but particularly in the brain, and resulting in an increased plaque load by

(a) reducing the β-amyloid plaque load, particularly by reducing the plaque area and plaque volume by at least 10%, particularly by at least 13%, more particularly by at least 20%, even more particularly by at least 26%, but especially by at least 30% and more as compared to the untreated control; and/or,
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control;
in tissues and organs, but particularly in the brain, of an animal, particularly a mammal, but especially a human through administration of a compound of formula III according to the invention and as described herein before or a pharmaceutical composition comprising said compound in a pharmaceutically effective amount.

In another specific embodiment, the invention relates to a method of treating an animal, particularly a mammal or a human, suffering from an amyloid-associated condition, characterized by a loss of cognitive memory capacity with a compound of formula III or a therapeutic composition comprising a compound of formula III according to the invention and as further defined herein, which treatment leads to the retention of cognitive memory capacity and/or an increase in cognitive memory capacity and/or a restoration of cognitive memory capacity in an animal, particularly a mammal or a human.

In a specific embodiment, the invention relates to the use of a compound of formula I, particularly of formula II, particularly of formula III as described herein, or a pharmaceutical composition comprising said compound in a pharmaceutically effective amount for the treatment of an animal, particularly a mammal, but especially a human or for the manufacture of a medicament for use in such a treatment, wherein plaque area and plaque volume is reduced by more than 10%, particularly by at least 13%, more particularly by at least 20%, even more particularly by at least 26%, but especially by at least 30% and more as compared to the untreated control, particularly in form of a pharmaceutical composition together with one or more pharmaceutically acceptable diluents or carriers therefore.

In still another embodiment, the invention relates to the use of a compound of formula I, particularly of formula II, particularly of formula III as described herein, or a pharmaceutical composition comprising said compound in a pharmaceutically effective amount for the treatment of an animal, particularly a mammal, but especially a human or for the manufacture of a medicament for use in such a treatment for retarding the increase of amyloid load to a level below that expected with normal progression of the disease, particularly to a level of at least 20%, more particularly to a level of at least 30%, even more particularly to a level of at least 50%, but especially to a level of at least 55% and up to 60% or more, particularly in form of a pharmaceutical composition together with one or more pharmaceutically acceptable diluents or carriers therefore.

The invention further relates to the use of a compound of formula I, particularly of formula II, particularly of formula III as described herein or a pharmaceutical composition comprising said compound in a pharmaceutically effective amount, for the treatment of a disease or condition in an animal, particularly a mammal, but especially a human, or for the manufacture of a medicament for use in such a treatment of a disease or condition, which is caused by or associated with the formation of β-amyloid plaques in tissues and organs, but particularly in the brain, of said animal, particularly said mammal, but especially said human, particularly a diseases or condition selected from the group consisting of neurological disorders such as Alzheimer's Disease (AD) and diseases or conditions characterized by a loss of cognitive memory capacity such as, for example, mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and others, including macular degeneration, drusen-related optic neuropathy and cataract due to beta-amyloid deposition, but especially Alzheimer's disease, or to a method of preparing a medicament to be used in such a treatment, particularly in form of a pharmaceutical composition together with one or more pharmaceutically acceptable diluents or carriers therefore.

In a specific embodiment, the invention relates to the use of a compound of formula particularly of formula II, particularly of formula III as described herein, or a pharmaceutical composition comprising said compound in a pharmaceutically effective amount for the treatment of an animal, particularly a mammal, but especially a human or for the manufacture of a medicament for use in such a treatment, for retaining cognitive memory capacity and/or increasing cognitive memory capacity and/or restoring cognitive memory capacity in an animal, particularly a mammal or a human.

In another specific embodiment of the invention, the compound of formula I, particularly of formula II, particularly of formula III or a pharmaceutical composition comprising said compound in a pharmaceutically effective amount, is administered orally.

The present invention relates to a method for reducing the β-amyloid plaque load in tissues and organs, but particularly in the brain, of an animal, particularly a mammal, but especially a human using a compound of formula I, particularly a compound of formula II, but especially a compound of formula III as disclosed herein before.

The invention also relates to a method for inhibiting the formation of β-amyloid plaques in tissues and organs, but particularly in the brain, of an animal, particularly a mammal, but especially a human using a compound of formula I, particularly a compound of formula II, but especially a compound of formula III.

The invention also relates to a method for retarding the increase of amyloid load in tissues and organs, but particularly in the brain, of an animal, particularly a mammal, but especially a human to a level below that expected with normal progression of the disease using a compound of formula I, particularly a compound of formula II, but especially a compound of formula III.

The compound according to formula I, particularly a compound of formula II, but especially a compound of formula III may be administered directly to a mammal, particularly a human patient, in need of such a treatment or, particularly, in form of a pharmaceutical composition together with one or more pharmaceutically acceptable diluents or carriers therefore.

In particular, a compound according to formula I, particularly a compound of formula II, but especially a compound of formula III or a pharmaceutical composition comprising said compounds, is administered orally or by intraperitoneal injection.

Preferably, the pharmaceutical composition according to the invention comprising a compound according to formula I, particularly a compound of formula II, but especially a compound of formula III, is provided in unit a dosage form such as tablets, pills, capsules, powders, granules, lozenges, sterile parenteral solutions or suspensions, metered aerosol or liquid sprays, drops, ampoules, autoinjector devices or suppositories for administration by oral, intranasal, sublingual, intraocular, transdermal, parenteral, rectal, vaginal, inhalation or insufflation means. Alternatively, the composition may be presented in a form suitable for application once a week, once every two weeks, once every three weeks, once every four week, etc; for example, as a slow release formulation.

The compound according to the present invention and as described herein before, particularly a compound of formula I, particularly a compound of formula II, but especially a compound of formula III, and pharmaceutically acceptable salts or hydrates thereof, can be prepared in a physiologically acceptable formulation and may comprise a pharmaceutically acceptable carrier, diluent and/or excipient using known techniques. Such compositions typically comprise a therapeutically effective amount of any of the compounds described herein above, and a pharmaceutically acceptable carrier. Preferably, the effective amount is an amount effective to reduce the β-amyloid plaque load or to inhibit the formation of β-amyloid plaques, or to retard the increase of amyloid load to a level below that expected with normal progression of the disease, in the brain of an animal, particularly a mammal, but especially a human. Suitable pharmaceutical carriers, diluents and/or excipients are well known to those skilled in the art.

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. Except insofar as any conventional media or agent is incompatible with the active compound, use thereof in the compositions is contemplated.

Solid carriers/diluents include, but are not limited to, a gum, a starch (e.g., corn starch, pregelatinized starch), a sugar (e.g., lactose, mannitol, sucrose, dextrose), a cellulosic material microcrystalline cellulose), an acrylate (e.g., polymethylacrylate), calcium carbonate, magnesium oxide, talc, or mixtures thereof. For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

A diluent may include, for example, phosphate buffered saline solutions, water, emulsions such as oil/water emulsions, various types of wetting agents, sterile solutions, etc. or microcrystalline cellulose.

The resulting pharmaceutical composition may contain other additives on demand, and, for example, a binder (e.g., starch, gum arabic, carboxymethyl cellulose, hydroxypropyl cellulose, crystalline cellulose etc.), a lubricant (e.g., magnesium stearate, talc etc.), a disintegrant (e.g., croscarmellose sodium; carboxymethyl cellulose calcium, talc etc.) and the like, and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof.

Binders (e.g., acacia, corn starch, gelatinee, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatine to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

Formulation of the compound according to formula I, particularly a compound of formula II, but especially a compound of formula III according to the invention can be accomplished according to standard methodology know to those skilled in the art.

Supplementary active compounds can also be incorporated into the pharmaceutical composition according to the invention.

After mixing various of the above-mentioned ingredients, the resulting mixture is formulated into a dosage form suitable for administration, particularly for oral administration.

The compound according to formula I, particularly a compound of formula II, but especially a compound of formula III and the pharmaceutical composition comprising said compound according to formula I, particularly a compound of formula II, but especially a compound of formula III of the present invention may be administered to a subject in the form of a solid, liquid or aerosol at a suitable, pharmaceutically effective dose. Examples of solid compositions include tablets, creams, and implantable dosage units. Tablets may be administered orally. Therapeutic creams may be administered topically. Implantable dosage units may be administered locally, or may be implanted for systematic release of the therapeutic composition, for example, subcutaneously. Examples of liquid compositions include formulations adapted for injection intramuscularly, subcutaneously, intravenously, intra-arterially, and formulations for topical and intraocular administration. Examples of aerosol formulations include inhaler formulations for administration to the lungs.

The compound according to formula I, particularly a compound of formula II, but especially a compound of formula III and the pharmaceutical composition comprising said compound according to formula I, particularly a compound of formula II, but especially a compound of formula III of the present invention may be administered by standard routes of administration. In general, the composition may be administered by topical, oral, rectal, nasal, interdermal, intraperitoneal, or parenteral (for example, intravenous, subcutaneous, or intramuscular) routes.

Administration may be parenterally, eg intravenously. Preparations for parenteral administration include sterile aqueous or non-aqueous solutions, suspensions and emulsions. Non-aqueous solvents include without being limited to it, propylene glycol, polyethylene glycol, vegetable oil such as olive oil, and injectable organic esters such as ethyl oleate. Aqueous solvents may be chosen from the group consisting of water, alcohol/aqueous solutions, emulsions or suspensions including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose) and others. Preservatives may also be present such as, for example, antimicrobials, anti-oxidants, chelating agents, inert gases, etc.

Administration will generally be orally. Dosage forms for or administration particularly comprise capsules, tablets, fine granules, granules, dry syrup and the like, and may be produced according to a method known per se. Preparations for oral administration can be combined with an oral, non-toxic, pharmaceutically acceptable, inert carrier such as but not limited to, lactose, starch, sucrose, glucose, methyl cellulose, magnesium stearate, dicalcium phosphate, calcium sulfate, mannitol, and sorbitol; for oral administration in liquid form, the oral drug components can be combined with any oral, non-toxic, pharmaceutically acceptable inert carrier such as, but not limited to, ethanol, glycerol, and water. Moreover, when desired or necessary, suitable binders, lubricants, disintegrating agents, and coloring agents can also be incorporated into the mixture. Suitable binders include, but not limited to, starch, gelatine, natural sugars such as, but not limited to, glucose or beta-lactose, corn sweeteners, natural and synthetic gums such as acacia, tragacanth, or sodium alginate, carboxymethylcellulose, polyethylene glycol, and waxes. Lubricants used in these dosage forms include sodium oleate, sodium stearate, magnesium stearate, sodium benzoate, sodium acetate, and sodium chloride. Disintegrants include, but are not limited to, starch, methyl cellulose, agar, bentonite, and xanthan gum.

Capsules may be prepared by filling standard two-piece hard gelatine capsules with powdered active ingredient, lactose, cellulose, and magnesium stearate.

Soft Gelatine capsules may be prepared by injecting by means of a positive displacement pump a mixture of active ingredient in a digestible oil such as soybean oil, cottonseed oil or olive oil into gelatine to form soft gelatine capsules containing the active ingredient. The capsules should be washed and dried.

Tablets may be prepared by conventional procedures so that the dosage unit, for example comprises active ingredient, colloidal silicon dioxide, magnesium stearate, microcrystalline cellulose, starch and lactose. Appropriate coatings may be applied to increase palatability or delay absorption.

Suspension may be prepared for oral and/or parenteral administration such as to contain finely divided active ingredient, sodium carboxymethyl cellulose, sodium benzoate, sorbitol solution, U.S.P., and vanillin or other palatable flavoring.

The pharmaceutical composition may further comprise proteinaceous carriers such as, for example, serum albumin or immunoglobulin, particularly of human origin. Further biologically active agents may be present in the pharmaceutical composition of the invention dependent on the intended use.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, the compound according to formula I, particularly a compound of formula II, but especially a compound of formula III and the pharmaceutical composition comprising said compound according to formula I, particularly a compound of formula II, but especially a compound of formula III according to the invention may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

It is well know to those skilled in the pertinent art that the dosage of the compound according to formula I, particularly a compound of formula II, but especially a compound of formula III and the pharmaceutical composition comprising said compound according to formula I, particularly a compound of formula II, but especially a compound of formula III according to the invention will depend on various factors such as, for example, the condition of being treated, the particular composition used, and other clinical factors such as weight, size, sex and general health condition of the patient, body surface area, the particular compound or composition to be administered, other drugs being administered concurrently, and the route of administration.

One factor determining the dosage regime to be applied is the bioavailability of the compound according to the invention after administration.

The bioavailability of the compounds according to the invention, particularly of a compound according to formula I, particularly a compound of formula II, but especially a compound of formula III can be determined by measuring the concentration of said compound in various tissues and body fluids such as brain, blood, serum, plasma, CSF, etc. These bioavailability studies can be used to determine the extent of central exposure of the experimental compound.

The experimental compound, particularly a compound according to formula I, particularly a compound of formula II, but especially a compound of formula III, can be quantified by standard methods known in the art such as, for example, UV-detection of appropriate HPLC fractions as described previously (Dusci et al., 2002). The mean elimination half life of a compound according to formula II is approx. 12 h after oral gavage. Peak plasma levels are achieved after approximately 3 h, which is perfectly in line with published data (Homan at al., 1987).

From the results obtained in the present invention it is evident that the compound according to formula II is capable of penetrating the blood-brain barrier, to an extent sufficient to exploit its pharmacological potential. At a dose of 100 mg/kg approx 0.5% of the plasma concentration was measured in the brains of 4 months old double transgenic mice and about 1% of the plasma concentration was measured in the brains of 8 months old single transgenic mice.

For the compound of formula III approx. 5% of the plasma concentration could be detected in the brains of 4 months old double transgenic mice as compared to about 11% of the plasma concentration in the brain of 8 months old single transgenic mice.

It was further shown within the scope of the present invention that the compound according to formula II and formula III, respectively, enters the CSF of 4 months old double transgenic mice to the extent of about 5% of the plasma concentration, as compared to about 9.5% that could be found in the CSF of human volunteers (i.e. 4 ng/mL; Jaup and Blomstrand, 1980).

The compound of formula III was shown to enter the CSF of 4 months old double transgenic mice to the extent of 20% of the plasma concentration. These observations are in line with results obtained in non-transgenic rats, where at 3 h or 6 h after i.p. administration of 50 mg/kg, a constant fraction of about 25% of the plasma concentration can be detected in CSF.

These data suggest that the compound of formula III is enriched in the brain to a certain extent.

It is shown in the present invention that the concentration of the compound according to the present invention and as described herein, but particularly of a compound of formula I, particularly a compound of formula II, but especially a compound of formula III in the brain and the CSF, respectively, is sufficiently high to exploit its pharmacological potential.

In particular, the concentration in the brain and the CSF, respectively, is such as to allow

(a) reducing the β-amyloid plaque load, particularly the plaque area and plaque volume by at least 10%, particularly by at least 13%, more particularly by at least 20%, even more particularly by at least 26%, but especially by at least 30% and more as compared to the untreated control; and/or,
(b) inhibiting the formation of β-amyloid plaques; and/or
(c) retarding the increase of amyloid load, particularly to a level below that expected with normal progression of the disease, particularly to a level of at least 20%, more particularly to a level of at least 30%, even more particularly to a level of at least 50%, but especially to a level of at least 55% and up to 60% or more as compared to the untreated control.

Based on an in vivo Alzheimer model represented by a very aggressive double transgenic mouse model for cerebral amyloidosis (Radde et al., 2006) expressing both KM670/671NL mutated human APP and L166P mutated human PS1 under the Thy-1 promoter (Radde et al., 2005) it could be shown that the compounds according to the invention are capable of substantially reducing the β-amyloid plaque load in the brain.

Transgenic (Tg) mice over-expressing human amyloid precursor protein (APP) are suitable models to study the influence of drugs on amyloid production, clearance, sequestration and deposition. The mice used within the scope of the present invention (APP751S/L) develop plaques consisting of amyloid depositions in early age, starting at 3 to 4 months and severity of the brain pathology correlates with increasing age.

The mentioned Tg hAPP751SL animals (former name TASD41) consecutively over-express human APP751 with the London (V7171) and the Swedish (K670M/N671L) mutations under the regulatory control of the neuronal tissue specific murine-Thy-1 promoter. The Thy-1 promoter ensures high expression in neurons mainly the brain and only little in the periphery. Due to the London mutation high levels of β-amyloid 1-42 are expressed all over the brain but mainly in cortex and hippocampus. Because the mutations introduced in this APP Tg mouse model are the same as the ones associated with FAD, it may be argued that this model might be more relevant to inherited than sporadic forms of AD. However, it is worth noting that in both sporadic and FAD the same upstream event (β-amyloid 1-42 accumulation) plays a central role in the pathogenesis of synaptic dysfunction and CAA. Thus, the findings in this model are likely translatable for both forms of AD.

To examine the potential of the experimental compounds according to the invention, daily p.o. doses were given aver an extended period of time. Daily administration of 50 mg/kg of the compound according to formula III and of 100 mg/kg of the compound according to formula II for the duration of one month at various stages between months 1 and 5 after birth of test animals led to substantial reductions of β-amyloid plaque load as was demonstrated by stereological analysis of stained brain sections.

These observations were supported by results obtained by staining of corresponding stereological brain sections from compound- and vehicle treated APPPS1 mice for microglia and astrocytes, which showed that these neuroinflammatory markers behaved in a similar fashion as β-amyloid plaque load.

The results obtained with the animal model suggest that treatment of the experimental animals with the compound of formula II and formula III; respectively, retarded the increase of amyloid load to about 55% and about 60% of that expected with normal progression of the model.

These results were confirmed by independent staining experiments with a different antibody against β-amyloid. Very similar results were obtained, reproducing the individual reductions observed in stained sections.

Plaque volume and area was shown to be about 26% and 13% smaller in APPPS1 mice (month 4-5) treated with the compound of formula III and the compound of formula II, respectively, as compared to respective vehicle-treated controls.

The compounds according to the present invention, particularly a compound according to formula I, particularly a compound of formula II, but especially a compound of formula III, were further shown to be capable of retaining or increasing cognitive memory capacity but, particularly, of restoring the cognitive memory capacity of an animal, particularly a mammal or a human, suffering from memory impairment by administering said compound to an animal, particularly a mammal or a human.

This could be demonstrated in the present application by exposing the transgenic APP mice to a Morris Water Maze task as described in the Examples. In the Morris Water Maze test system, the cognitive capabilities of an experimental animal are tested. In particular, the ability of the experimental animal to find a hidden platform using visual cues is measured for a fixed period of time performing several trials a day. By comparing of the learning curves, the cognitive capabilities can be determined and possible drug effects can be evaluated.

The results of the overall performance expressed as escape latency (time) in seconds as swimming path (length) in meters show that all treatment groups were able to learn and improve their performance in the Morris Water Maze. Mice treated with 20 mg/kg of the compound according to formula II and to a lesser extent mice treated with 1 mg/kg of the compound according to formula II, showed a comparable escape latency to the non transgenic vehicle treated mice.

Further, the results obtained in the probe trial, where the platform has been taken out of the pool and the number of crossings over the former target position as well as the abidance in the target quadrant has been counted for a given period of time, confirmed the escape latency results. Transgenic animals treated with the compound according to formula II in a concentration of 1 mg/kg crossed the former target position significantly more often than animals from the control group.

In still another embodiment of the invention, the compound according to formula I, particularly the compound of formula II, but especially the compound of formula III as described herein before, or a composition comprising said compound, may be administered in combination with another biologically active substance or compound or with a composition comprising said substance or compound, particularly in combination with a biologically active substance or compound that acts complementary with the compound according to the invention such as a compound according to formula I, particularly a compound of formula H, but especially a compound of formula ill as described herein before, in the treatment of a condition associated with the formation and deposition of β-amyloid plaques in tissues and organs, but particularly in the brain, of an animal, particularly a mammal, but especially a human, particularly a compound selected from the group consisting of compounds against oxidative stress, anti-apoptotic compounds, metal chelators, inhibitors of DNA repair, 3-amino-1-propanesulfonic acid (3APS), 1,3-propanedisulfonate (1,3PDS), α-secretase activators, β- and γ-secretase inhibitors, tau proteins, neurotransmitter, β-sheet breakers, attractants for amyloid beta clearing/depleting cellular components, inhibitors of N-terminal truncated amyloid beta including pyroglutamated amyloid beta 3-42, anti-inflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine, M1 agonists and other drugs including any amyloid or tau modifying drug and nutritive supplements such as, for example, vitamin B12, cysteine, a precursor of acetylcholine, lecithin, cholin, Ginkgo biloba, acetyl-L-carnitine, idebenone, propentofylline, or a xanthine derivative, together with an antibody according to the present invention and, optionally, a pharmaceutically acceptable carrier and/or a diluent and/or an excipient and procedures for the treatment of diseases.

In particular, the compound according to formula I, particularly a compound of formula II, but especially a compound of formula III may be used together with an acetylcholine esterase inhibitor, such as tacrine, donepezil, rivastigmine and galanthamine in form of a composition. In a specific embodiment, a complementary composition is provided comprising the compound according to formula I, particularly a compound of formula II, but especially a compound of formula III and the acetylcholine esterase inhibitor in an amount that results in a complementary action of the compounds. Acetylcholine esterase inhibitors are widely used for the palliative treatment of patients suffering from Alzheimer's disease and related disorders. All marketed acetylcholine esterase inhibitors, however, produce severe side effects in patients, such as nausea, vomiting, diarrhea, anorexia, weight loss and, in the case of tacrine. These side effects are due to the higher levels of acetylcholine in peripheral organs, such as the stomach. These side effects can be effectively suppressed by peripherally acting acetylcholine receptor antagonists, such as a compound according to formula I, particularly a compound of formula II, but especially a compound of formula III leaving the central effects of the acetylcholine esterase inhibitors untouched.

The active ingredients comprised within the therapeutical compositions according to the invention and as described herein before including the compounds according to formula I, particularly a compound of formula II, but especially a compound of formula III may be administered together as a single composition or separately in form of two or more distinct compositions each containing one or more active ingredients. Furthermore, if administered separately in form of two or more distinct compositions, said distinct compositions may be administered at the same time or successively.

When the target is located in the brain, certain embodiments of the invention provide for the compound according to formula I, particularly the compound of formula II, but especially the compound of formula III and the pharmaceutical composition comprising said compound according to formula I, particularly said compound of formula II, but especially said compound of formula III of the present invention to traverse the blood-brain barrier. Certain neurodegenerative diseases are associated with an increase in permeability of the blood-brain barrier, such that the antibody or active fragment thereof can be readily introduced to the brain. When the blood-brain barrier remains intact, several art-known approaches exist for transporting molecules across it, including, but not limited to, physical methods, lipid-based methods, and receptor and channel-based methods.

Physical methods of transporting a compound across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9: 398-406 (2002)) and implanting a delivery device in the brain (see, e.g., Gill at al., Nature Med. 9: 589-595 (2003); and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Lipid-based methods of transporting the compound according to formula I, particularly the compound of formula II, but especially the compound of formula III and the pharmaceutical composition comprising said compounds of the present invention across the blood-brain barrier include, but are not limited to, encapsulating the compound according to the invention in liposomes that are coupled to antibody binding fragments that bind to receptors on the vascular endothelium of the blood-brain barrier (see, e.g., U.S. Patent Application Publication No. 2002/0025313), and coating the compound according to the invention in low-density lipoprotein particles (see, e.g., U.S. Patent Application Publication No. 2004/0204354) or apolipoprotein E (see, e.g., U.S. Patent Application Publication No. 2004/0131692).

Receptor and channel-based methods of transporting the compound according to the invention across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating antibodies with a transferrin and modulating activity of the one or more transferrin receptors (see, e.g., U.S. Patent Application Publication No. 2003/0129186), and cationizing the compound according to the invention (see, e.g., U.S. Pat. No. 5,004,697).

It will be understood that various details of the presently disclosed subject matter may be changed without departing from the scope of the presently disclosed subject matter. Furthermore, the foregoing description is for the purpose of illustration only, and not for the purpose of limitation.

### EXAMPLES

The following examples will further illustrate some of the embodiments of the present invention without, however, being considered in any way limiting for the invention. In light of the present disclosure and the general level of skill in the art, those of skill will appreciate that the following Examples are intended to be exemplary only and that numerous changes, modifications, and alterations can be employed without departing from the scope of the presently claimed subject matter

### A General Methodology

### A1. 1^{st} Study

### A1.1 Western Blots an Immunostaining

Monoclonal anti-PARP antibody was purchased from BD BioScience (Cat #556 362; clone C2-10). Secondary anti-mouse alkaline phosphatase conjugate was purchased from Sigma (Cat #A9316). NBT/BCIP-Western blot detection reagents came from Roche Diagnostics (Cat. #1681451), Western Lightening CDP-Star chemoluminescence detection kit was supplied by Perkin-Elmer (Cat. #NEL616001KT). For anti-PARP Western blotting experiments proteins were separated on 10% polyacrylamide gels and blotted onto nitrocellulose. Blots were blocked with 5% skimmed milk powder in Tris buffered saline containing 0.1% Tween-20 (TBST); anti-PARP antibody was incubated over night at 4° C. using a 1:1000 dilution in milk powder TBST. Blots were subsequently washed 3 times using TBS-T. Second antibody was used at a dilution of 1:1000 for NBT/BCIP detection and 1:5000 for CDP-Star detection. Gels from various Sir-2 containing fractions were blotted onto nitrocellulose membranes and visualized accordingly. For Sir-2 staining the following antibodies were used: primary Ab: anti-Sir 2 (Upstate, Biomol 07-131; Lot: 22073); 1:5000 in 5% BSA/1×TBST; secondary Ab: anti-Rabbit PE(A-0545) 1:1000 in 5% BSA/1×TBST; To detect specifically human APP in Western blots mouse monoclonal antibody 6E10 that recognizes residue 1-17 of human Aβ was used (Signet, Dedham, Mass.).

### A1.2 Bioavailability Experiments

The bioavailability of the compounds was determined in male Lewis rats (207+/−9 g). the a AC91 compound was formulated in 0.5% carboxymethylcellulose in water for oral application. AC-92 was prepared in DMSO and diluted in sterile phosphate buffered saline (final DMSO concentration 1.0%). AC91 was administered by oral gavage and AC-92 by intra peritoneal injection. Animals were sacrificed at 3 and 6 hours after dosing via lethal narcosis. Blood was sampled via cardiac puncture. Serum was prepared by allowing whole blood to stand at 4° C. for 60 min; plasma was prepared using heparin as the anti-coagulant. CSF was collected via the foramen magnum immediately after sacrifice. Brain material was collected by opening of the skull and simple excision of the right cortex. Samples were snap frozen using liquid nitrogen immediately after collection. All procedures were conducted in conformity with applicable German and EU laws on animal experimentation and the study was approved by a government appointed ethics committee.

### A1.3 Transgenic Model for Cerebral Amyloidosis: APPPS1 Experiments

The transgenic model and corresponding serological analysis of brain sections was provided by Prof. Mathias Jucker, Department of Cellular Neurology, Hertie-Institute for Clinical Brain Research University of Tübingen, Otfried-Müller Strasse 27, D-72076 Tübingen, Germany. APPPS1 transgenic mice express both KM670/671NL mutated human APP and L166P mutated human PS1 under the Thy-1 promoter element (Radde et al., 2005). They were treated with the compounds from the age of 126 days after birth (DAB) to 158 DAB. Mice were treated with either the vehicle (0.5% methyl cellulose, 0.25% lecithin, 0.1% microcrystalline cellulose) or a commercial formulation of AC91 (100 mg/kg) suspended in 0.5% WN methyl cellulose, 0.25% WN lecithin once daily by gavage at a time corresponding to the first third of the resting period after the dark cycle. On completion of the dosing period, animals were sacrificed by lethal narcosis followed by collection of blood by cardiac puncture and recovery of brain material for sectioning and extraction of drug and relevant peptides. Samples were snap frozen using liquid nitrogen immediately after collection. All procedures were conducted in conformity with applicable German and EU laws on animal experimentation and the study was approved by a government appointed ethics committee. Brains were removed and postfixed at 4° C. in 4% PFA, dehydrated in 30% sucrose, and frozen. Serial coronal serial 40 μm sections were cut with a microtome and collected in cryoprotectant (30% glycerol, 45% ethylene glycol in PBS) and stored at −20° C. until use.

Free-floating sections were processed for immunohistochemistry as described elsewhere (Stalder et al., 2005). Briefly, sections were washed in TBS and blocked with 3% goat or donkey serum (Vector Laboratories Inc., Burlingame, Calif.) in 0.3% Triton-X-100 (Fisher, Fair Lawn, N.J.). The sections were incubated overnight with primary antibodies at 4° C. in 2% serum and 0.3% Triton-X-100, washed three times with TBS and incubated for 3 hours with biotin-conjugated secondary antibodies. After repeated TBS washing, sections were stained by complexing with SG blue (Vectastain ABC elite kit; Vector Laboratories). Sections were mounted on precleaned glass microscope slides (Superfrost® Plus; Langenbrinck, Teningen, Germany), dehydrated with an alcohol series, cleared in xylene and coverslipped in a xylene soluble mounting medium (Pertex®, medite GmbH, Burgdorf, Germany). Amyloid load was estimated on every 12th section throughout the entire neocortex.

### A2 2^{nd} Study

A second study was designed to evaluate the efficacy of two experimental compounds (AC-91, AC-92) on behavioral markers using 7 months (±2 weeks) old female APP Tg and nTg mice.

Therefore, mice were treated for 33 days and in the end of the treatment period behavior was evaluated in the Morris Water Maze and additionally a Object Recognition Task.

### A2.1 Animals

Female Tg and nTg mice with a C57BL/6×DBA background and an age of 7 months (±2 week) were randomly assigned to treatment groups 1 to 9 (n=20 for groups 3 to 7, n=15 for groups 1, 2, 8 and 9). Animals were subjected to administration of vehicle, AC-91 and AC-92 beginning at 7 months of age and continued for up to 33 days with daily oral application. All animals which were used for the present study had dark eyes and were likely to perceive the landmarks outside the MWM pool. However, it had to be excluded that seeing abilities of an animal were poor, which was controlled in the visible platform training, the so called pretest, before treatment start for all animals including reserves enclosed to the study. In case a seeing handicap for a specific animal would have been affirmed, the mouse would have been excluded from the study.

### A2.2 Materials

ACI-91 dihydrochloride hydrate was obtained from Tocris Cookson Ltd., Bristol BS11 9XJ, UK and delivered by Anawa Trading SA

ACI-92, free base, was synthesized and provided by ProteoSys, Mainz, Germany.

### A2.3 Treatment

130 (plus 8 reserves) transgenic and 30 (plus 3 reserves) non-transgenic mice were allocated to 8 groups received either the experimental compounds (dosage AC-91 and dosage AC-92) or vehicle (2×PBS and Tween 80, respectively). Compounds or vehicle were administered via oral gavage in a daily volume of 10 ml/kg/b.w. for 33 days.

### A2.4 Analysis

Determination of ACI-91 and ACI-92 in mouse plasma, CSF and brain homogenate samples was done by HPLC-MS/MS by Quality Assistance SA, Technoparc de Thudinie 2, B-6536 Donstiennes, Belgium.

### A2.5. Behavioral Testing

### A2.5 1 Behavioral Test in the Object Recognition Task

The Object Recognition task is a behavioral paradigm to measure visual recognition memory, which is evolutionarily conserved in species including humans and rodents and which requires the hippocampus. The object recognition task was performed as described elsewhere (Dewachter et al. 2002). Briefly, mice were habituated for 1 hour to a Plexiglas box (48×48 cm) with dark vertical walls and a translucent floor dimly illuminated by a lamp placed underneath the box. The next day, the animals were placed in the same box and submitted to a 10 minute acquisition trial. During this trial, mice were individually placed into a Plexiglas box in the presence of two objects A and C. The time spent exploring object A (when the animal's snout was directed toward the object at a distance ˜1 cm) was measured. During a 10 minute retention trial (second trial), which was performed 3 hours later, the object C was replaced by a novel object B. Therefore, the novel object B was placed together with the familiar object (object A) in the box.

The time (tA and tB) the animal spends exploring the two objects was recorded. The recognition index (RI), defined as the ratio of the time spent exploring the novel object over the time spent exploring both objects [(tB/(tA tB))×100] was used to measure non-spatial memory. Behavior was video tracked.

### A2.5.2 Morris Water Maze (MWM)

The Morris Water Maze task was conducted in a black circular pool of a diameter of 100 cm. Tap water was filled in with a temperature of 22±1° C. and the pool was virtually divided into four sectors. A transparent platform (8 cm diameter) was placed about 0.5 cm beneath the water surface. During the whole test session, except the pretest, the platform was located in the southwest quadrant of the pool.

One day before the 4 days lasting training session animals had to perform a so called “pre-test” (two 60 sec lasting trials) to ensure that the seeing abilities of each animal were normal. Only animals that fulfilled this task were enclosed to the MWM testing.

In the MWM task, each mouse had to perform three trials on four consecutive days. A single trial lasted for a maximum of one minute. During this time, the mouse had the chance to find the hidden, diaphanous target. If the animal could not find a “way” out of the water, the investigator guided to or placed the mouse on the platform. After each trial mice were allowed to rest on the platform for 10-15 sec.

During this time, the mice had the possibility to orientate in the surrounding. Investigations took place under dimmed light conditions, to prevent the tracking system from negative influences (Kaminski; PCS, Biomedical Research Systems). On the walls surrounding the pool, posters with black, bold geometric symbols (e.g. a circle and a square) were fixed which the mice could use the symbols as landmarks for their orientation.

One swimming group per trial consisted of five to six mice, so that an intertrial time of about five to ten minutes was ensured. For the quantification of escape latency (the time [second]−the mouse needed to find the hidden platform and therefore to escape from the water), of pathway (the length of the trajectory [meter] to reach the target) and of the abidance in the goal quadrant a computerized tracking system was used. The computer was connected to a camera placed above the centre of the pool. The camera detected the signal of the light emitting diode (LED), which was fixed with a little hairgrip on the mouse's tail.

Twenty-four hours after the last trial on day 4 the mice had to fulfil a so-called probe trial. At this time, the platform was removed from the pool and during the one-minute probe trial; the experimenter counted the number of crossings over the former target position. Additionally the abidance in this quadrant as well as the three other quadrants was calculated. Through out this trial a mouse could not get any, howsoever natured, clue from the platform.

### A2.6. Statistics

Means and standard error of means (SEM) were calculated for all measured parameters.

Behavioral data were compared by means of a parametric or non-parametric ANOVA followed by a Newman Keuls or a Dunn's Multiple Comparison test in dependence of data distribution.

Differences were calculated by a parametric ANOVA followed by a Newman Keuls multiple comparison post-hoc test or by a non-parametric Kruskal Wallis ANOVA followed by a Dunn's Multiple comparison test, if Gaussian distribution was missing. Not to underestimate differences in the ANOVA due to the fact that several groups had similar means, group differences were evaluated by parametric unpaired, two tailed test, if data turned out to be normally distributed; otherwise, groups were compared by means of a non-parametric Mann Whitney U-test. Outliers within a group were detected by Grubbs test and were excluded from all calculations.

### B Experiments

### B1. 1^{st} Study

### B1.1 Bioavailability Studies in Non-Transgenic Rats

To determine the extent of central exposure, bioavailability studies were undertaken. In one set of experiments, 16 rats were given 50 mg/day AC-91 or AC-92 and either killed after 3 h or 6 h. Plasma and cerebrospinal fluid (CSF) of 64 animals were collected and AC-91 and AC-92 were quantified by UV-detection of appropriate HPLC fractions at 244 nm and 330 nm as described previously (Dusci et al., 2002). The mean elimination half life of AC-91 is approx. 12 h after oral gavage. Peak plasma levels were achieved after 3 h, which is perfectly in line with published data (Homon et al., 1987). 3 h after oral administration of 50 mg/kg AC-91, about 900 fMoles/μl plasma can be detected, which declines to approx. 200 fMoles/μl after 6 h. For the main metabolite des-methyl-AC-91, the corresponding values were 370 and 180 fMoles/μl, respectively. No AC-91 or dm-AC-91 was detected in CSF under conditions described, which is in line with reports of the blood brain barrier (BBB) permeability of AC-91 in rodents. This situation is slightly different in humans, where about 10% of the AC-91 compound available in plasma moves into the CSF (Jaw and Blomstrand, 1980). Concerning the des-piperazinyl metabolite AC-92, only about 20 fMoles/μl of the compound was found 3 h after oral administration of 50 mg/kg AC-91 in plasma, but a similar amount in the CSF. These amounts decrease slightly in the plasma after 6 h, but more than triple to about 75 fMoles/μl in the CSF. So, AC-92 is enriched in the brain to a certain extent. AC-92 itself permeates the BBB fairly well: at 3 h or 6 h, a constant fraction of about 25% of the AC-92 compound measured in plasma after i.p. administration of 50 mg/kg can be detected in CSF.

### B1.2 Determination of for Plaque Load, Plaque Volume and Area in APPPS1 Experiments

In vivo experiments were performed using a very aggressive double transgenic mouse model for cerebral amyloidosis (Radde et al., 2006). APPPS1 transgenic mice expressing both KM670/671NL mutated human APP and L166P mutated human PS1 under the Thy-1 promoter element (Radde et al., 2005) were treated with the compounds from the age of 126 days after birth (DAB) to 158 DAB. Mice were treated as described in A3. above. On completion of the dosing period, samples were taken from the animals and snap frozen using liquid nitrogen as reported herein previously (see Section A3. above).

Daily p.o. administration of 50 mg/kg AC-92 or 100 mg/kg AC-91 for the duration of one month at various stages between months 1 and 5 after birth of test animals, led to substantial reductions of β-amyloid plaque load. Staining of corresponding stereological brain sections from AC-91- and vehicle treated APPPS1 mice for microglia and astrocytes showed that these neuroinflammatory markers behaved in a similar fashion as β-amyloid plaque load. Based on stereological analysis of stained sections (n=13 to 18 sections per animal), vehicle treated mice in the current experiment exhibited a cortical amyloid load of 0.82% at month 3 and 3.27% at month 5. In the model, plaque load in the brain is known to increase roughly exponentially with age (Radde et al., 2005). Based on these deposition kinetics, there is an estimated increase in plaque load of ca. 0.45% between months 2 and 3 and of 1.01% between months 4 and 5, respectively. Hence background plaque load in APPPS mice at months 2 and 4 is estimated to be ca. 0.37% and 2.26%, thus providing conditions of increasing severity of cerebral amyloidosis. These conditions should be suitable to provide insight, whether initial formation of plaques or downstream processes reversing existing plaque loads are involved in corresponding drug effects.

Under conditions chosen, a full arrest of plaque deposition after drug administration would, e.g., result in 5 month plaque loads in the order of 2.3%, and a 50% reduction in plaque deposition would result in plaque loads in the order 2.8%. The corresponding values for 3-month old animals are 0.4 and 0.6%. AC-92-treated animals after 3 months, and AC-91-treated mice at month 5 had amyloid loads of 0.61 and 2.86% suggesting that treatment retarded the increase of amyloid load to 55% and 60% of that expected with normal progression of the model. Based on the plaque loads of individual sections (13 to 18 sections per animal, 5-8 animals per group), the differences between the corresponding groups were significant with p-values <0.0001, whereas groups differed at p<0.03 and 0.09, respectively, based on animal mean plaque loads. Some of the remaining second halves of the brains were used independently for Western blots stained with a different antibody against β-amyloid. Very similar results were obtained, reproducing the individual reductions observed in stained sections. Brain sections of AC-92-treated animals after 3 months, and AC-91-treated mice at month 5 were stained with a polyclonal antibody to ionized calcium binding adapter molecule 1 (Iba1) as a marker for microglia. Consecutive serial sections were stained with a polyclonal antibody to glial fibrillary acidic protein (GFAP).

Plaque volume and area are about 26% and 13% smaller in AC-92-treated (month 2-3) and AC-91 treated APPPS1 mice (month 4-5) as compared to respective vehicle-treated controls.

### B2. 2^{nd} Study

### B2.1 ACI-91 and ACI-92 Levels in Transgenic Mice

The amounts of ACI-91 and of ACI-92 in plasma, CSF and in brain homogenates is determined after treatment of hAPP single transgenic mice (JSW, Graz) and hAPP-PS1 double-transgenic mice (Synovo, Tübingen), respectively, for 33 days with doses of 1, 5, 20 and 100 mg/kg of ACI-91 and 50 mg/kg ACI-92 and doses of 100 mg/kg of ACI-91 and 50 mg/kg ACI-92, respectively.

The results show that ACI-91 does penetrate the blood-brain barrier, to a small extent. At a dose of 100 mg/kg of ACI-91 less than 0.5% of the plasma concentration was measured in the brains of 4 months old double transgenic mice. Compare: At a dose of 100 mg/kg of ACI-91 less than 1% of the plasma concentration was measured in the brains of 8 months old single transgenic mice.

ACI-91 metabolism to ACI-92 in 4 months old double transgenic mice was not detectable. In comparison, ACI-91 is metabolized to ACI-92 in plasma to an extent of about 0.5% in 8 months old single transgenic mice.

ACI-92 enters the brains of 4 months old double transgenic mice to an extent of about 5% of the plasma concentration. Compare: ACI-92 enters the brain of 8 months old single transgenic mice to an extent of 11% of the plasma concentration.

ACI-91 enters the CSF of 4 months old double transgenic mice to the extent of about 5% of the plasma concentration, comparable to the 9.5% into the CSF of human volunteers (i.e. 4 ng/mL; Jaup and Blomstrand, 1980).

ACI92 enters the CSF of 4 months old double transgenic mice to the extent of 20% of the plasma concentration.

### B2.2 Evaluation of the Efficacy of Two Experimental Compounds (AC-91, AC-92) on Behavioral, Biochemical and Histological Markers

Transgenic (Tg) mice over-expressing human amyloid precursor protein (APP) are suitable models to study the influence of drugs on amyloid production, clearance, sequestration and deposition. The mice used for the present study (APP751S/L) develop plaques consisting of amyloid depositions in early age, starting at 3 to 4 months and severity of the brain pathology correlates with increasing age.

The mentioned Tg hAPP751SL animals (former name TASD41) consecutively over-express human APP751 with the London (V7171) and the Swedish (K670M/N671L) mutations under the regulatory control of the neuronal tissue specific murine-Thy-1 promoter. The Thy-1 promoter ensures high expression in neurons mainly the brain and only little in the periphery. Due to the London mutation high levels of βamyloid 1-42 are expressed all over the brain but mainly in cortex and hippocampus. Because the mutations introduced in this APP Tg mouse model are the same as the ones associated with FAD, this model might be more relevant to inherited than sporadic forms of AD. However, it is worth noting that in both sporadic and FAD the same upstream event (β-amyloid 1-42 accumulation) plays a central role in the pathogenesis of synaptic dysfunction and CAA. Thus, the findings in this model are likely translatable for both forms of AD.

### B2.2.1 General Observations

In total 171 female hAPP Tg and nTg mice with an age of 6.5 months at treatment start were enclosed to study. From these mice 16 animals (14 Tg and 2 nTg mice) died due to unknown reason before the treatment period was finished. With a death rate >10% the present study lies clearly below the average death rate of hAPP mice used in 23 comparable studies (see Appendix 7). In general, animals well tolerated the treatment with either the vehicles (2×PBS and Tween 80) or the both test items AC-91 (in four different concentrations) and B. People performing the treatment did not report any obvious pain reactions during or after the applications. Furthermore, no negative influence on the development of the body weight during the treatment period could be seen (see Appendix 7), then even the weight loss of treatment group I (ntg Tween 80) was not significant between treatment start and end. Wet weight of the left hemisphere was also not influenced by any treatment.

### B2.2.2 Behavioral Results

Results of the behavioral investigations are shown in the FIGS. 1 to 4. The results obtained in the Object Recognition Task (ORT) are shown in the Appendix. Due to the fact that the tg and ntg mice were not significantly different in RI, this memory test failed the validation and it is therefore not for memory testing in this Tg mouse line (results are shown in Appendix 6). Results in the Morris Water Maze—revealing cognitive functions from the two treatment groups at the end of the 33 days lasting treatment are shown in FIGS. 1 to 4. Over a period of 4 days, the ability to find a hidden platform using visual cues is measured performing 3 trials a day. By comparing of the learning curves, the cognitive abilities can be checked and possible drug effects can be evaluated.

FIG. 1 shows the results of the overall performance as escape latency (time) in seconds and FIG. 2 shows the results as swimming path (length) in meters. Data are presented as mean of each group on each of the four days. In general, it can be stated that all treatment groups were able to learn and improve their performance in the Morris Water Maze. No significant differences occurred between the different treatments group. However, mice treated with 20 mg/kg AC-91 and to a lesser extent 1 mg/kg, showed a comparable escape latency to the ntg vehicle treated mice. Mice treated with the other concentration of AC-91 or with Compounds B showed a weak performance similar to that observed in the historic tg group.

FIG. 3 shows the results obtained in the probe trial. During this trial, the platform has been taken out of the pool and the number of crossings over the former target position as well as the abidance in the target quadrant has been counted for 30 seconds. Transgenic animals treated with the AC-91 in the concentrations 1 mg/kg crossed the former target position significantly (p<0.05) more often than animals from the Tween 80 vehicle group (upper graph FIG. 3).

FIG. 4 shows the improvement in time and length between trial 1 on day 1 (first trial in the Morris Water Maze training) and trial 3 on day 4 (last trial). This parameter did not reveal significant group differences although mice treated with AC-91, except dose 100 mg/kg, showed similar results as ntg mice.

### B2.3. Summary of Effects and Conclusion

Effects that could be observed after treatment:

When compared to the historic group AC-91 led to an improvement in performing the Morris Water Maze task; the escape latency of mice treated with the concentrations 1 and 20 mg/kg was reduced relatively to the historic group. In the probe trial, mice treated with AC-91 in the concentrations 1 mg/kg significantly (p<0.05) more often crossed over the former platform position than animals treated with vehicle group (Tween 80). Mice treated with AC-92 showed no difference to the Tween 80 mice. Further dose escalation to 100 mg/kg didn't improve memory performance.

### LIST OF ABBREVIATIONS

Aβ beta amyloid
Aβ1-40, Aβ1-42 beta amyloid peptide fragments 1-40, 1-42
AC-91 pirenzepine
AC-92 LS-75
APP amyloid precursor protein
AD Alzheimer's disease
b.w. body weight
C57BL/6×DBA background of Tg and nTg mice
CAA Cerebral amyloid angiopathy
CSF cerebrospinal fluid
ELISA enzyme-linked immunosorbent assay
FAD Familiar Alzheimer's disease
hAPP human amyloid precursor protein
JSW CNS JSW CNS Research, Forschungslabor GmbH
MWM Morris Water Maze
n number
n.a. not applicable
n.m. not measurable
ORT New Object Recognition task
nTg non-transgenic
p.o. per orally
PBS Phosphate buffer saline
RT or r.t. room temperature
SDS sodium dodecyl sulfate
SEM Standard error of means
TBS TRIS buffered saline
Tg transgenic

### REFERENCE LIST

Dusci L J, Peter H L, Fellows L M, Ilett K F (2002) Determination of olanzapine in plasma by high-performance liquid chromatography using ultraviolet absorbance detection. J Chromatogr B Analyt Technol Biomed Life Sci 773:191-197.
Gill et al., Nature Med, 9: 589-595 (2003)
Hammer R, Berrie C P, Birdsall N J, Burgen A S, Hulme E C (1980) pirenzepine distinguishes between different subclasses of muscarinic receptors. Nature 283:90-92.
Homon C A, Esber H J, Zavorskas P, Tanswell P, Farina P R (1987) A selective radioimmunoassay for the determination of pirenzepine in plasma and urine. Ther Drug Monit 9:236-242.
Jaup B H, Biomstrand C (1980) Cerebro-spinal fluid concentrations of pirenzepine after therapeutic dosage. Scand J Gastroenterol Suppl 66:35-37.
McGeer et al., in: Alzheimer Disease and Associated Disorders, Vol 8(3), pp 149-158; 1994, Raven Press Ltd, New York
Neuwelt, E. A., Implication of the Blood-Brain Barrier and its Manipulation, Vols 1 & 2, Plenum Press, N.Y. (1989)
Papanastassiou et al., Gene Therapy 9: 398-406 (2002)
Radde R, Belmont T, Käser S A, Coorriaraswamy J, Jäggi F, Gengler S, Haass C, Staufenbiel M, Czech C, Ghetti B, Hölscher C, Mathews P M, Jucker M (2006) A42-driven cerebral amyloidosis in APPPS1 transgenic C57BL/6 mice reveals early and robust pathology EMBO Rep. 2006 September; 7(9):940-6.
Stalder A K, Ermini F, Bondolfi L, Krenger W. Burbach G J, Deller T, Coomaraswamy J, Staufenbiel M, Landmann R, Jucker M (2005) Invasion of hematopoietic cells into the brain of amyloid precursor protein transgenic mice. J Neurosci 25:11125-11132.
Suh S W, Aoyama K, Chen Y, Garnier P, Matsumori Y, Gum E, Liu J, Swanson R A (2003) Hypoglycemic neuronal death and cognitive impairment are prevented by poly(ADP-ribose) polymerase inhibitors administered after hypoglycemia. J Neurosci 23:10681-10690.

### PATENT LITERATURE

### WO 2006/008118

U.S. Pat. No. 4,522,811
U.S. Pat. No. 5,112,596
U.S. Pat. No. 5,268,164
U.S. Pat. No. 5,506,206
U.S. Pat. No. 5,686,416
U.S. Pat. No. 5,004,697
U.S. Patent Appl. Publication No. 2002/0038086
U.S. Patent Appl. Publication No, 2002/0025313
U.S. Patent Appl. Publication No. 2004/0204354
U.S. Patent Appl. Publication No. 2004/0131692
U.S. Patent Appl. Publication No. 2002/0065259
U.S. Patent Appl. Publication No. 2003/0162695
U.S. Patent Appl. Publication No. 2005/0124533
U.S. Patent Appl. Publication No. 2005/0089473
U.S. Patent Appl. Publication No. 2003/0073713
U.S. Patent Appl. Publication No. 2003/0129186

1-52. (canceled)

53. A compound of formula I 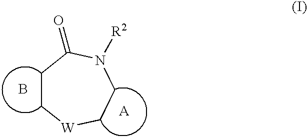
wherein A and B are five- or six-membered rings optionally containing at least one heteroatom selected from N, S and O, wherein the rings are optionally mono- or polysubstituted with halo, e.g. F, Cl, Br, or I, C1-C4-(halo)-alkyl, C1-C4-(halo)-alkoxy, amino, C1-C4-alkyl-amino, or di(C1-C4-alkyl)amino,
W is S, O, NR1 or CHR1
R1 is hydrogen, Y or COY,
R2 is hydrogen or C1-C4-(halo)-alkyl, and
Y is C1-C6 (halo)alkyl, or C3-C8 cyclo-(halo)alkyl, wherein the alkyl or cycloalkyl group is optionally substituted with a five- or six-membered ring optionally containing at least one heteroatom selected from N, S and O, and wherein the ring is optionally mono- or poly-substituted with halo, C1-C4-(halo)alkyl, C1-C4(halo)alkoxy, amino, C1-C4-alkyl amino, di(C1-C4-alkyl)amino or Z,
wherein Z is a C1-C6 (halo) alkyl group ω-substituted with a group N(R4)2, wherein each R4 is independently hydrogen, C1-C8 alkyl, or CO—C1-C8-alkyl or wherein both R4 together from a five- or six-membered ring optionally containing at least one further heteroatom selected from N, S and O, wherein the ring is optionally mono- or polysubstituted with halo, C1-C4(halo)-alkyl and C1-C4(halo)alkoxy, or of a salt or derivative thereof, or a pharmaceutical composition comprising said compound
in a pharmaceutically effective amount for reducing the β-amyloid plaque load as compared to the untreated control; and/or
inhibiting the formation of β-amyloid plaques; and/or
retarding the increase of amyloid load, particularly to a level below that expected with normal progression of the disease as compared to the untreated control in the brain of an animal.

54. The compound of claim 53 or a pharmaceutical composition comprising said compound, wherein the compound of formula I comprises a cyclic group A and a cyclic group B, 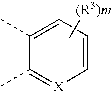
wherein X is N or CR3,
R3 is in each case independently halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2.

55. The compound of claim 53, or a pharmaceutical composition comprising said compound, wherein the compound of formula I comprises a cyclic group A, 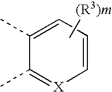
wherein X is N
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2.

56. The compound of claim 53 or a pharmaceutical composition comprising said compound, wherein the compound of formula I comprises a cyclic group B 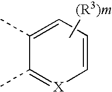
wherein X is CH
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2.

57. The compound of claim 53 or a pharmaceutical composition comprising said compound, wherein
W is NR1, and
R1 is COY and
Y is —(CHR7)q-R8
wherein R7 is hydrogen, halo or C1-C4-(halo)alkyl,
q is an integer of 1-4, and preferably 1 and
R8 is a five- or six-membered ring optionally containing at least one heteroatom, wherein the ring is optionally mono- or polysubstituted with C1-C4-(halo)alkyl or a ω-amino-substituted alkyl group Z as defined above.

58. The compound of claim 53 or a pharmaceutical composition comprising said compound, wherein
W is NR1, and
R1 is COY and
Y is —(CHR7)q-R8
wherein R7 is hydrogen or C1-C4-alkyl,
q is an integer of 1-4, and preferably 1 and
R8 is a six-membered ring containing at least one N, wherein the ring is mono- or polysubstituted with C1-C4-(halo)alkyl.

59. The compound of claim 53 or a pharmaceutical composition comprising said compound, wherein the compound of formula I comprises a cyclic group A, 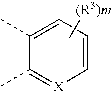
wherein X is N
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2; and a cyclic group B, 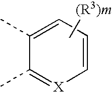
wherein X is CH
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2; and wherein
W is NR1
R1 is COY and
Y is —(CHR7)q-R8
wherein R7 is hydrogen, halo or C1-C4-(halo)alkyl,
q is an integer of 1-4, and preferably 1 and
R8 is a five- or six-membered ring optionally containing at least one heteroatom, wherein the ring is optionally mono- or polysubstituted with C1-C4(halo)alkyl or a ω-amino-substituted alkyl group Z as defined above.

60. The compound of claim 53 or a pharmaceutical composition comprising said compound, wherein the compound of formula I comprises a cyclic group A, 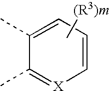
wherein X is N
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2; and a cyclic group B, 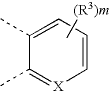
wherein X is CH
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2; and wherein
W is NR1
R1 is COY and
Y is —(CHR7)q-R8
wherein R7 is hydrogen or C1-C4-alkyl,
q is an integer of 1-4, and preferably 1 and
R8 is a six-membered ring containing at least one N, wherein the ring is mono- or polysubstituted with C₁-C₄-(halo)alkyl.

61. The compound of claim 53 or a pharmaceutical composition comprising said compound, wherein
W is NR1
R1 is hydrogen
the cyclic group A and B is 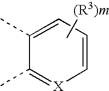
wherein X is N or CR3, and
R3 is in each case independently halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2.

62. The compound of claim 53 or a pharmaceutical composition comprising said compound, wherein
W is NR1
R1 is hydrogen
the cyclic group B is 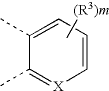
wherein X is CR3, and
R3 is in each case independently halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2.

63. The compound of claim 53 or a pharmaceutical composition comprising said compound, wherein
W is NR1
R1 is hydrogen
the cyclic group A is 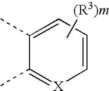
wherein X is N, and
R3 is halo, C1-C4-(halo)-alkyl, C1-C4-(halo)-alkyl, C1-C4-(halo)-alkoxy, amino, C1-C4-alkyl-amino, or di(C1-C4-alkyl)amino, and
m is an integer of 0-2.

64. The compound of claim 53 or a pharmaceutical composition comprising said compound, wherein
W is NR1
R1 is hydrogen
the cyclic group A is 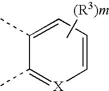
wherein X is N, and
R3 is C1-C4-(halo)-alkyl, and
m is an integer of 0-2; and
wherein the cyclic group B is 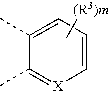
wherein X is CH
R3 is in each case C1-C4-(halo)-alkyl, and
m is an integer of 0-2.

65. The compound of claim 53 or a pharmaceutical composition comprising said compound, which is a compound of formula II.

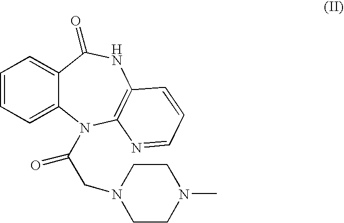

66. The compound of claim 53 or a pharmaceutical composition comprising said compound, which is a compound of formula III.

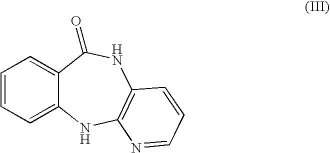

67. The compound of claim 53 or a pharmaceutical composition comprising said compound, wherein the plaque area and plaque volume is reduced by more than 13% as compared to the untreated control.

68. The compound of claim 53 or a pharmaceutical composition comprising said compound, wherein plaque area and plaque volume is reduced by more than 20% as compared to the untreated control.

69. The compound of claim 53 or a pharmaceutical composition comprising said compound, wherein plaque area and plaque volume is reduced by more than 26% as compared to the untreated control.

70. The compound of claim 53 or a pharmaceutical composition comprising said compound, wherein the increase of amyloid load is retarded to at least 55% of that expected with normal progression of the disease.

71. The compound of claim 53 or a pharmaceutical composition comprising said compound, wherein the increase of amyloid load is retarded to at least 60% of that expected with normal progression of the disease.

72. The compound of claim 53 or a pharmaceutical composition comprising said compound, wherein reducing the β-amyloid plaque load, inhibiting the formation of β-amyloid plaques and/or retarding the increase of amyloid load in the brain of an animal leads to a reduction and/or amelioration of the effects of a disease or condition caused by or associated with the formation and deposition of β-amyloid plaques in the brain.

73. The compound of claim 53 or a pharmaceutical composition comprising said compound, wherein said disease or condition is selected from the group consisting of neurological disorders including Alzheimer's Disease (AD) and diseases or conditions characterized by a loss of cognitive memory capacity including Lewy body dementia, mild cognitive impairment (MCI), Down's syndrome, hereditary cerebral hemorrhage with amyloidosis (Dutch type); the Guam Parkinson-Dementia complex; as well as other diseases which are based on or associated with amyloid-like proteins including progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, macular degeneration, drusen-related optic neuropathy and cataract due to beta-amyloid deposition.

74. The compound of claim 53 or a pharmaceutical composition comprising said compound in a pharmaceutically effective amount, for the treatment in an animal of a condition caused by or associated with the formation of β-amyloid plaques in tissues and organs and resulting in an increased plaque load, or for use in such a treatment, by
reducing the β-amyloid plaque load, particularly by reducing the plaque area and plaque volume by at least 10% as compared to the untreated control; and/or
inhibiting the formation of β-amyloid plaques; and/or
retarding the increase of amyloid load, particularly to a level below that expected with normal progression of the disease as compared to the untreated control;
in the brain of the animal.

75. The compound of claim 53 or a pharmaceutical composition comprising said compound in a pharmaceutically effective amount, for retaining or increasing cognitive memory capacity in an animal suffering from memory impairment.

76. The compound of claim 53 or a pharmaceutical composition comprising said compound in a pharmaceutically effective amount, for restoring the cognitive memory capacity of an animal suffering from memory impairment.

77. The pharmaceutical composition comprising the compound of claim 53 and a biologically active substance or compound, particularly at least one compound selected from the group consisting of: compounds against oxidative stress, anti-apoptotic compounds, metal chelators, inhibitors of DNA repair, 3-amino-1-propanesulfonic acid (3APS), 1,3-propanedisulfonate (1,3PDS), α-secretase activators, β- and γ-secretase inhibitors, tau proteins, neurotransmitter, β-sheet breakers, attractants for amyloid beta clearing/depleting cellular components, inhibitors of N-terminal truncated amyloid beta including pyroglutamated amyloid beta 3-42, anti-inflammatory molecules, atypical antipsychotics including clozapine, ziprasidone, risperidone, aripiprazole or olanzapine or cholinesterase inhibitors (ChEIs) including tacrine, rivastigmine, donepezil, and/or galantamine, M1 agonists, amyloid or tau modifying drugs and nutritive supplements including vitamin B12, cysteine, a precursor of acetylcholine, lecithin, cholin, Ginkgo biloba, acetyl-L-carnitine, idebenone, propentofylline, and a xanthine derivative.

78. The pharmaceutical composition of claim 53 further comprising a cholinesterase inhibitor (ChEIs).

79. The pharmaceutical composition of claim 78 wherein the cholinesterase inhibitor (ChEIs) selected from the group consisting of tacrine, rivastigmine, donepezil, and galantamine.

80. A method of using a compound of claim 53 for (a) reducing the β-amyloid plaque load, and/or (b) inhibiting the formation of β-amyloid plaques and/or (c) retarding the increase of amyloid load in the brain of an animal.

81. The method of claim 80 of using an effective amount of a compound of formula II 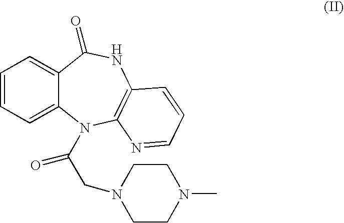
for (a) reducing the β-amyloid plaque load, and/or (b) inhibiting the formation of β-amyloid plaques and/or (c) retarding the increase of amyloid load in the brain of an animal using a compound of formula II.

82. The method according to claim 80 of using an effective amount of a compound of formula III 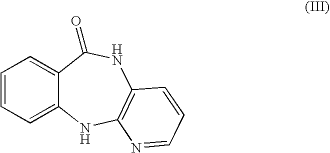
for (a) reducing the β-amyloid plaque load, and/or (b) inhibiting the formation of β-amyloid plaques and/or (c) retarding the increase of amyloid load in the brain of an animal.

83. The method of claim 80 wherein
(a) the β-amyloid plaque load, particularly the plaque area and plaque volume is reduced by at least 10% as compared to the untreated control; and/or
(b) the formation of β-amyloid plaques is inhibited; and/or
(c) the increase of amyloid load is retarded, particularly to a level below that expected with normal progression of the disease as compared to the untreated control;
in the brain of an animal.

84. The method of claim 80 for the treatment of a disease or condition in an animal, wherein the disease of condition is associated with the formation of β-amyloid plaques in the brain.

85. The method of claim 80, wherein the diseases or condition caused by or associated with the formation of β-amyloid plaques in the brain is a disease or condition selected from the group consisting of neurological disorders such as Alzheimer's Disease (AD) and diseases or conditions characterized by a loss of cognitive memory capacity including Lewy body dementia, mild cognitive impairment (MCI), Down's syndrome, hereditary cerebral hemorrhage with amyloidosis (Dutch type); the Guam Parkinson-Dementia complex; as well as other diseases which are based on or associated with amyloid-like proteins including progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, macular degeneration, drusen-related optic neuropathy and cataract due to beta-amyloid deposition.

86. The method of claim 80 for treatment of a condition of memory impairment by retaining or increasing cognitive memory capacity in an animal suffering from memory impairment.

87. The method of claim 80 for treatment of a condition of memory impairment by restoring the cognitive memory capacity of an animal suffering from memory impairment.

88. The method of claim 87, wherein the diseases or condition is Alzheimer's disease.

89. The method of claim 80, wherein the compound is administered orally.

90. The method of claim 80, wherein the compound is used as a pro-drug.

91. A method of (a) reducing the β-amyloid plaque load, and/or (b) inhibiting the formation of β-amyloid plaques and/or (c) retarding the increase of amyloid load in the brain of an animal, by administering to the animal, a compound or a pharmaceutical composition of claim 53.

92. The method according to claim 91, wherein said compound is a compound of formula II or III.

93. The method of claim 91, wherein
(a) the β-amyloid plaque load, particularly the plaque area and plaque volume is reduced by at least 10%, as compared to the untreated control; and/or
(b) the formation of β-amyloid plaques is inhibited; and/or
(c) the increase of amyloid load is retarded, particularly to a level below that expected with normal progression of the disease, as compared to the untreated control;
in the brain of the animal, by administering to an animal, a compound of formula I.

94. The method of claim 92, wherein the compound is a compound of formula II.

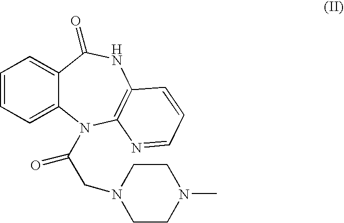

95. The method of claim 92, wherein the compound is a compound of formula III.

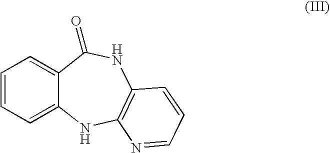

96. A method for treating, a condition caused by or associated with the formation of β-amyloid plaques in the brain and resulting in an increased plaque load in an animal by
(a) reducing the β-amyloid plaque load, by reducing the plaque area and plaque volume by at least 10%, as compared to the untreated control; and/or
(b) inhibiting the formation of β-amyloid plaques; and/or
(c) retarding the increase of amyloid load to a level below that expected with normal progression of the disease;
in the brain of the animal, through administration of a compound or a pharmaceutical composition of claim 53.

97. The method of claim 96, wherein the compound is a compound of formula II.

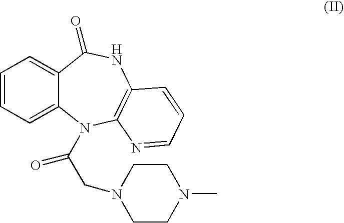

98. The method of claim 96, wherein the compound is a compound of formula III.

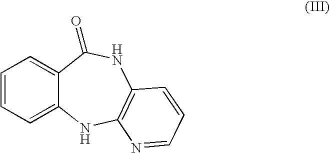

99. The method of claim 96 for treating a condition in an animal caused by or associated with the formation of β-amyloid plaques in the brain and resulting in an increased plaque load, wherein said disease or condition is selected from the group consisting of neurological disorders including Alzheimer's Disease (AD) and diseases or conditions characterized by a loss of cognitive memory capacity including mild cognitive impairment (MCI), Lewy body dementia, Down's syndrome, hereditary cerebral hemorrhage with amyloidosis (Dutch type); the Guam Parkinson-Dementia complex; as well as other diseases which are based on or associated with amyloid-like proteins including progressive supranuclear palsy, multiple sclerosis; Creutzfeld Jacob disease, Parkinson's disease, HIV-related dementia, ALS (amyotropic lateral sclerosis), Adult Onset Diabetes; senile cardiac amyloidosis; endocrine tumors, and macular degeneration.

100. A method for retaining or increasing cognitive memory capacity in an animal suffering from memory impairment by administering to an animal a compound of formula I according to claim 53, and/or a pharmaceutically effective metabolite thereof or a pharmaceutical composition comprising said compound and/or a pharmaceutically effective metabolite thereof.

101. The method of claim 101, wherein said metabolite is a compound of claim 67.

102. A pharmaceutical composition for suppressing side effects resulting from the use of acetylcholine esterase inhibitors for the treatment of patients suffering from Alzheimer's disease comprising a compound according to formula I, formula II, or formula III, and an acetylcholine esterase inhibitor together with a pharmaceutically acceptable carrier and/or a diluent and/or an excipient.

103. A pharmaceutical composition according to claim 102, wherein the acetylcholine esterase inhibitor is a compound selected from the group consisting of tacrine, donepezil, rivastigmine and galanthamine.

104. A pharmaceutical composition according to claim 102, wherein the compound according to formula I, formula II, or formula III, and the acetylcholine esterase inhibitor are provided in separate unit dosage forms.
